(19) United States
(12) Patent Application Publication
(10) Pub. No.: US 2017/0263874 A1
(43) Pub. Date: Sep. 14, 2017

(54) CARBON NANOTUBE COMPOSITE, SEMICONDUCTOR DEVICE AND METHOD FOR PRODUCING THE SAME, AND SENSOR USING THE SAME (AS AMENDED)

(71) Applicant: TORAY INDUSTRIES, INC., TOKYO (JP)

(72) Inventors: Kazuki Isogai, Otsu-shi, Shiga (JP); Seiichiro Murase, Otsu-shi, Shiga (JP); Hiroji Shimizu, Otsu-shi, Shiga (JP)

(73) Assignee: TORAY INDUSTRIES, INC., TOKYO (JP)

(21) Appl. No.: 15/529,282

(22) PCT Filed: Nov. 19, 2015
(86) PCT No.: PCT/JP2015/082518

§ 371 Date: May 24, 2017

(30) Foreign Application Priority Data
Nov. 26, 2014 (JP) 2014-238515

(51) Int. Cl. H01L 51/05; G01N 33/68; G01N 27/414; H01L 51/10; H01L 51/00

(52) U.S. Cl. CPC H01L 51/0541; C01B 31/0273; H01L 51/0558; H01L 51/105; H01L 51/0048; G01N 27/4146; G01N 27/4145; G01N 33/68; B82Y 15/00

(57) ABSTRACT

Provided is a CNT composite capable of achieving both high detection sensitivity and specific detection when used as a sensor. The carbon nanotube composite includes an aggregation inhibitor (A) and a blocking agent (B) attached to at least a portion of a surface.

### CROSS REFERENCE TO RELATED APPLICATIONS

This is the U.S. National Phase application of PCT/JP2015/082518, filed Nov. 19, 2015, which claims priority to Japanese Patent Application No. 2014-238515, filed Nov. 26, 2014, the disclosures of each of these applications being incorporated herein by reference in their entireties for all purposes.

### FIELD OF THE INVENTION

The present invention relates to a carbon nanotube composite, a semiconductor device and a method for producing the same, and a sensor using the same.

### BACKGROUND OF THE INVENTION

Semiconductor devices, such as transistors, memories, and capacitors, have been used in various electronic devices, such as displays and computers, utilizing their semiconducting properties. For example, utilizing the electrical characteristics of field-effect transistors (hereinafter referred to as FET), the development of IC tags and sensors has also been advanced. Among them, FET-type biosensors that detect a biological reaction using an FET have been actively studied for the reasons that labeling with a fluorescent substance or the like is not required, electrical signal conversion is fast, and connection to an integrated circuit is easy.

Conventionally, a biosensor using an FET, which is called “ion-sensitive FET sensor”, is configured such that a gate electrode is removed from an MOS (metal-oxide-semiconductor)-type FET, and an ion-sensitive membrane is deposited on an insulating film. Then, a biomolecular recognition substance is disposed on the ion-sensitive membrane, whereby such sensors are designed to function as various types of biosensors.

However, the application to an immunosensor or the like utilizing the antigen-antibody reaction, which requires high detection sensitivity, is technically limited in terms of detection sensitivity, and its practical use has not yet been achieved. In addition, the process of forming a film of an inorganic semiconductor, such as silicon, requires expensive production equipment and, thus, there is a problem in that cost reduction is difficult. Further, because the film production process is performed at an extremely high temperature, there is a problem in that the kind of material usable as the substrate is limited and, thus, a lightweight resin substrate, for example, cannot be used.

In recent years, for the purpose of solving the above problems with inorganic semiconductors such as silicon, FET sensors including a semiconductor layer formed by applying an organic compound solution have been developed. Among them, it is known that a coating-type FET sensor using carbon nanotubes (hereinafter referred to as CNT) having high mechanical/electrical characteristics has high detection sensitivity.

For example, a pH sensor prepared by dispersing CNTs in water using carboxymethylcellulose as an aggregation inhibitor, followed by spin-coating the dispersion to forma semiconductor layer, and a DNA sensor prepared by dispersing CNTs in heavy water using sodium dodecyl sulfate (SDS) as an aggregation inhibitor, followed by drop-casting the dispersion to form a semiconductor layer, are known (see, e.g., Non-Patent Documents 1 and 2). In addition, a sensor using CNTs covered with a film of a hydrophilic polymer, such as polyethylene glycol, has also been disclosed (see, e.g., Patent Document 1).

### PATENT DOCUMENT

Patent Document 1: JP 2006-505806 W

### NON-PATENT DOCUMENTS

Non-Patent Document 1: BIOCHIMICA ET BIOPHYSICA ACTA, Vol. 1830, (2013) 4353-4358

Non-Patent Document 2: JOURNAL OF AMERICAN CHEMICAL SOCIETY, 2007, Vol. 129, 14427-14432

### SUMMARY OF THE INVENTION

With the techniques described in Non-Patent Documents 1 and 2, because the surface of CNTs is not protected, it has been difficult to specifically detect the target protein. In addition, with the technique described in Patent Document 1, sensitivity improvement has been limited.

In light of the above problems, an object of the present invention is to provide a CNT composite capable of achieving both high detection sensitivity and specific detection when used as a sensor.

In order to solve the above problems, the present invention is configured as follows. That is, the present invention is directed to a carbon nanotube composite including an aggregation inhibitor (A) attached to at least a portion of the surface of carbon nanotubes, the carbon nanotube composite including a blocking agent (B) attached to at least a portion of the surface of carbon nanotubes.

The present invention is also directed to a semiconductor device including a substrate, a first electrode, a second electrode, and a semiconductor layer, the first electrode being spaced apart from the second electrode, the semiconductor layer being disposed between the first electrode and the second electrode, the semiconductor layer containing the carbon nanotube composite described above. The present invention is further directed to a sensor including the semiconductor device described above.

According to the present invention, a sensor that achieves both high detection sensitivity and specific detection can be provided.

### BRIEF DESCRIPTION OF THE DRAWINGS

FIG. 1 is a schematic cross-sectional view showing a semiconductor device according to an aspect of the present invention.

FIG. 2 is a schematic cross-sectional view showing a semiconductor device according to an aspect of the present invention.

FIG. 3 is a schematic plan view showing a sensor according to an aspect of the present invention.

FIG. 4A is a schematic plan view showing a sensor according to an aspect of the present invention.

FIG. 4B is a schematic cross-sectional view showing a sensor according to an aspect of the present invention.

FIG. 5A is a schematic plan view showing a sensor according to an aspect of the present invention.

FIG. 5B is a schematic cross-sectional view showing a sensor according to an aspect of the present invention.

FIG. 6 is a schematic cross-sectional view showing a sensor according to an aspect of the present invention.

FIG. 7 is a graph showing the value of the current flowing between the first electrode and the second electrode when BSA, IgE, and avidin are added to the semiconductor layer of a semiconductor device shown in an example of the present invention.

FIG. 8 is a graph showing the value of the current flowing between the first electrode and the second electrode when BSA, IgE, and avidin are added to the semiconductor layer of a semiconductor device shown in an example of the present invention.

### DETAILED DESCRIPTION OF EMBODIMENTS OF THE INVENTION

<Carbon Nanotube Composite>

In the carbon nanotube (hereinafter referred to as CNT) composite of the present invention, an aggregation inhibitor (A) and a blocking agent (B) are attached to at least a portion of the surface of carbon nanotubes. In addition, it is preferable that at least a portion of the CNT composite has at least one functional group selected from the group consisting of a hydroxyl group, a carboxy group, an amino group, a mercapto group, a sulfo group, a phosphonic acid group, an organic salt or inorganic salt thereof, a formyl group, a maleimide group, and a succinimide group.

The state in which an aggregation inhibitor and a blocking agent are attached to at least a portion of the surface of CNTs means the state in which the surface of CNTs is partially or completely covered with the aggregation inhibitor and the blocking agent. In this state, on the surface of CNTs, there may be an area overlappingly covered with both the aggregation inhibitor and the blocking agent. In addition, the state in which an organic compound (C) is attached to at least a portion of the surface of CNTs as described below means the state in which the surface of CNTs is partially or completely covered with the organic compound (C). In this state, on the surface of CNTs, there may be an area overlappingly covered with the aggregation inhibitor, the blocking agent, and the organic compound (C).

The reason why an aggregation inhibitor and a blocking agent can cover CNTs is presumably attributed to their hydrophobic interaction with CNTs. In addition, in the case where the aggregation inhibitor or the blocking agent has a conjugated structure, the reason is presumably attributed to an interaction caused by the overlapping of π-electron clouds derived from the conjugated structures of the aggregation inhibitor or blocking agent and the CNTs.

When CNTs are covered with an aggregation inhibitor or a blocking agent, the reflected color of CNTs becomes closer to the color of the aggregation inhibitor or the blocking agent than to the original color of non-covered CNTs. By observing the color change, whether CNTs are covered can be judged. Quantitatively, by elemental analysis such as X-ray photoelectron spectroscopy (XPS), the presence of an attached substance can be confirmed, and the weight ratio of the attached substance relative to CNTs can be measured.

In the CNT composite of an embodiment of the present invention, an aggregation inhibitor is attached to at least a portion of the surface of CNTs. Accordingly, CNTs can be uniformly dispersed in a solution without impairing the high electrical characteristics of CNTs. In addition, by a coating method using the solution having CNTs uniformly dispersed therein, a uniformly dispersed CNT film can be formed. As a result, high semiconducting properties can be achieved.

Methods for attaching an aggregation inhibitor to CNTs include:

(I) a method in which CNTs are added to a melted aggregation inhibitor and mixed;

(II) a method in which the aggregation inhibitor is dissolved in a solvent, and CNTs are added thereto and mixed;

(III) a method in which CNTs are pre-dispersed ultrasonically, etc., and the aggregation inhibitor is added thereto and mixed; and

(IV) a method in which the aggregation inhibitor and CNTs are added to a solvent, and the mixed system is mixed by ultrasonic irradiation.

In the present invention, any of these methods may be used, and it is also possible to use any combination of these methods.

In the CNT composite of an embodiment of the present invention, a blocking agent is attached to at least a portion of the surface of CNTs. Accordingly, the adsorption of non-target proteins onto CNTs can be prevented. As a result, specific detection of proteins is enabled.

In addition, in the CNT composite of the present invention, because an aggregation inhibitor is attached to at least a portion of the surface of CNTs, as compared with CNTs having no aggregation inhibitor attached thereto, the degree of decrease in detection sensitivity accompanying the attachment of a blocking agent to the surface of CNTs can be reduced. The reason for this is presumably that in the CNT composite of the present invention, the attachment of an aggregation inhibitor to at least a portion of the surface of CNTs has the effect of lessening the interaction between CNTs and the blocking agent.

Methods for attaching a blocking agent to CNTs include:

(I) a method in which CNTs are added to a melted blocking agent and mixed;

(II) a method in which the blocking agent is dissolved in a solvent, and CNTs are added thereto and mixed;

(III) a method in which CNTs are pre-dispersed ultrasonically, etc., and the blocking agent is added thereto and mixed;

(IV) a method in which the blocking agent and CNTs are added to a solvent, and the mixed system is mixed by ultrasonic irradiation;

(V) a method in which CNTs applied onto a substrate are immersed in a melted blocking agent; and

(VI) a method in which the blocking agent is dissolved in a solvent, and CNTs applied onto a substrate are immersed therein.

In the present invention, any of these methods may be used, and it is also possible to use any combination of these methods. In terms of detection sensitivity, it is preferable to use a method in which a blocking agent is attached to CNTs utilizing a solid-liquid reaction, such as (V) or (VI).

The aggregation inhibitor and the blocking agent may be the same or different compounds. In terms of detection sensitivity, it is preferable that they are different compounds.

The order of attaching an aggregation inhibitor and a blocking agent to CNTs is not particularly limited, but it is preferable that the aggregation inhibitor is attached, and then the blocking agent is attached.

(CNT)

As CNTs, single-wall CNTs composed of a single carbon film (graphene sheet) cylindrically wound, double-wall CNTs composed of two graphene sheets concentrically wound, and multi-wall CNTs composed of a plurality of graphene sheets concentrically wound are all usable. However, in order to obtain high semiconducting properties, it is preferable to use single-wall CNTs. CNTs can be obtained by an arc discharge method, a chemical vapor deposition method (CVD), a laser abrasion method, or the like.

In addition, it is more preferable that CNTs include 80 wt % or more of semiconducting CNTs, still more preferably 95 wt % or more of semiconducting CNTs. As a method for obtaining CNTs including 80 wt % or more of semiconducting CNTs, a known method may be used. Examples thereof include a method in which ultracentrifugation is performed in the presence of a density gradient agent, a method in which a specific compound is selectively attached to the surface of semiconducting or metallic CNTs, followed by separation utilizing the difference in solubility, and a method in which separation is performed by electrophoresis or the like utilizing the difference in electrical properties. Examples of a methods for measuring the semiconducting CNT content include calculation from the absorption area ratio of the visible-near infrared absorption spectrum and calculation from the Raman spectrum intensity ratio.

In the present invention, it is preferable that the length of CNTs is shorter than the distance between a first electrode and a second electrode in a semiconductor device or sensor to which the present invention is applied. Specifically, although this depends on the channel length, it is preferable that the average length of CNTs is 2 82 m or less, more preferably 1 μm or less. The average length of CNTs means the average of the lengths of randomly picked up 20 CNTs. A method for measuring the average length of CNTs is, for example, a method in which 20 CNTs are randomly picked up from an image obtained using an atomic force microscope, a scanning electron microscope, a transmission electron microscope, or the like, and their lengths are averaged.

Commercially available CNTs have a length distribution, and it may happen that CNTs longer than a distance between the electrodes are contained. Accordingly, it is preferable that a step of making CNTs shorter than the distance between electrodes is added. For example, a method in which CNTs are cut into the shape of short fibers by an acid treatment with nitric acid, sulfuric acid, or the like, an ultrasonic treatment, a freeze grinding method, or the like is effective. In addition, in terms of improving the purity, it is still more preferable to also use separation through a filter.

In addition, the diameter of CNTs is not particularly limited, but is preferably 1 nm or more and 100 nm or less, and more preferably 50 nm or less.

In the present invention, it is preferable to include a step of uniformly dispersing CNTs in a solvent and filtering the dispersion through a filter. By obtaining CNTs smaller than the pore size of the filter from the filtrate, CNTs shorter than between the electrodes can be efficiently obtained. In this case, it is preferable that the filter is a membrane filter. The pore size of the filter used for filtration should be smaller than the channel length, and is preferably 0.5 to 10 μm. Other methods for making CNTs shorter and smaller include an acid treatment and a freeze grinding treatment.

(Aggregation Inhibitor (A))

An aggregation inhibitor is a compound that is attached to the surface of CNTs and thus has the effect of suppressing the aggregation of CNTs in the medium.

The aggregation inhibitor is not particularly limited. Specific examples thereof include polyvinyl alcohol, celluloses such as carboxymethylcellulose, polyalkylene glycols such as polyethylene glycol, acrylic resins such as polyhydroxymethyl methacrylate, conjugated polymers such as poly-3-hexyl thiophene, polycyclic aromatic compounds such as an anthracene derivative and a pyrene derivative, and long-chain alkyl organic salts such as sodium dodecyl sulfate and sodium cholate.

In terms of the interaction with CNTs, those having a hydrophobic group such as an alkyl group or an aromatic hydrocarbon group or having a conjugated structure are preferable. Among them, those that are polymers are preferable, and conjugated polymers are particularly preferable. A conjugated polymer makes it possible to disperse CNTs uniformly in a solution without impairing the high electrical characteristics of CNTs, whereby even higher semiconducting properties can be achieved.

Examples of polymers include cellulose, carboxymethylcellulose, polyhydroxymethyl methacrylate, polyacrylic acid, alginic acid, sodium alginate, polyvinyl sulfonic acid, sodium polyvinyl sulfonate, polystyrene sulfonic acid, sodium polystyrene sulfonate, polyvinyl alcohol, and polyethylene glycol. The above polymers maybe used alone, and it is also possible to use two or more kinds of compounds. As the polymer, it is preferable to use a polymer composed of single monomers formed a line, but it is also possible to use a polymer obtained by the block copolymerization or random copolymerization of different monomer units. In addition, it is also possible to use a polymer obtained by graft polymerization.

Examples of conjugated polymers include, but are not particularly limited to, polythiophene polymers, polypyrrole polymers, polyaniline polymers, polyacetylene polymers, poly-p-phenylene polymers, and poly-p-phenylene vinylene polymers. As the conjugated polymer, it is preferable to use a polymer composed of single monomers formed a line, but it is also possible to use a polymer obtained by the block copolymerization or random copolymerization of different monomer units. In addition, it is also possible to use a polymer obtained by graft polymerization.

Among the above polymers and conjugated polymers, in the present invention, carboxymethylcellulose and polythiophene polymers, which can be easily attached to CNTs to form a CNT composite, are preferable, and it is particularly preferable to use a polythiophene polymer.

The above conjugated polymer does not necessarily have to have a high molecular weight, and may also be a linear conjugated oligomer. It is preferable that the molecular weight of the conjugated polymer is, as a number average molecular weight, 800 to 100,000.

Specific example structures of conjugated polymers having the above structure are as shown below. Incidentally, n in each structure represents the number of repeats and is within a range of 2 to 1,000. In addition, the conjugated polymer may be a homopolymer of each structure, or may also be a copolymer.

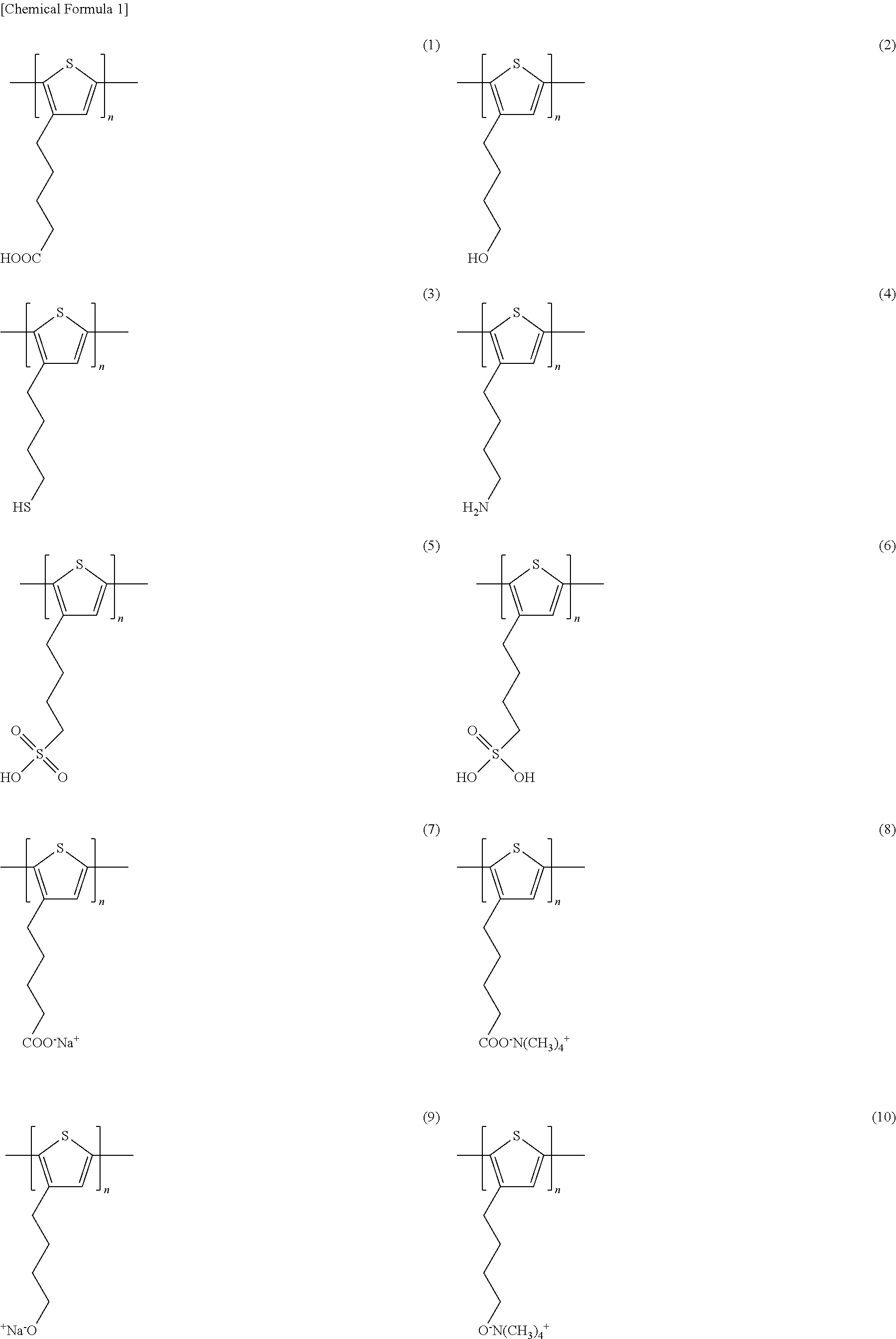

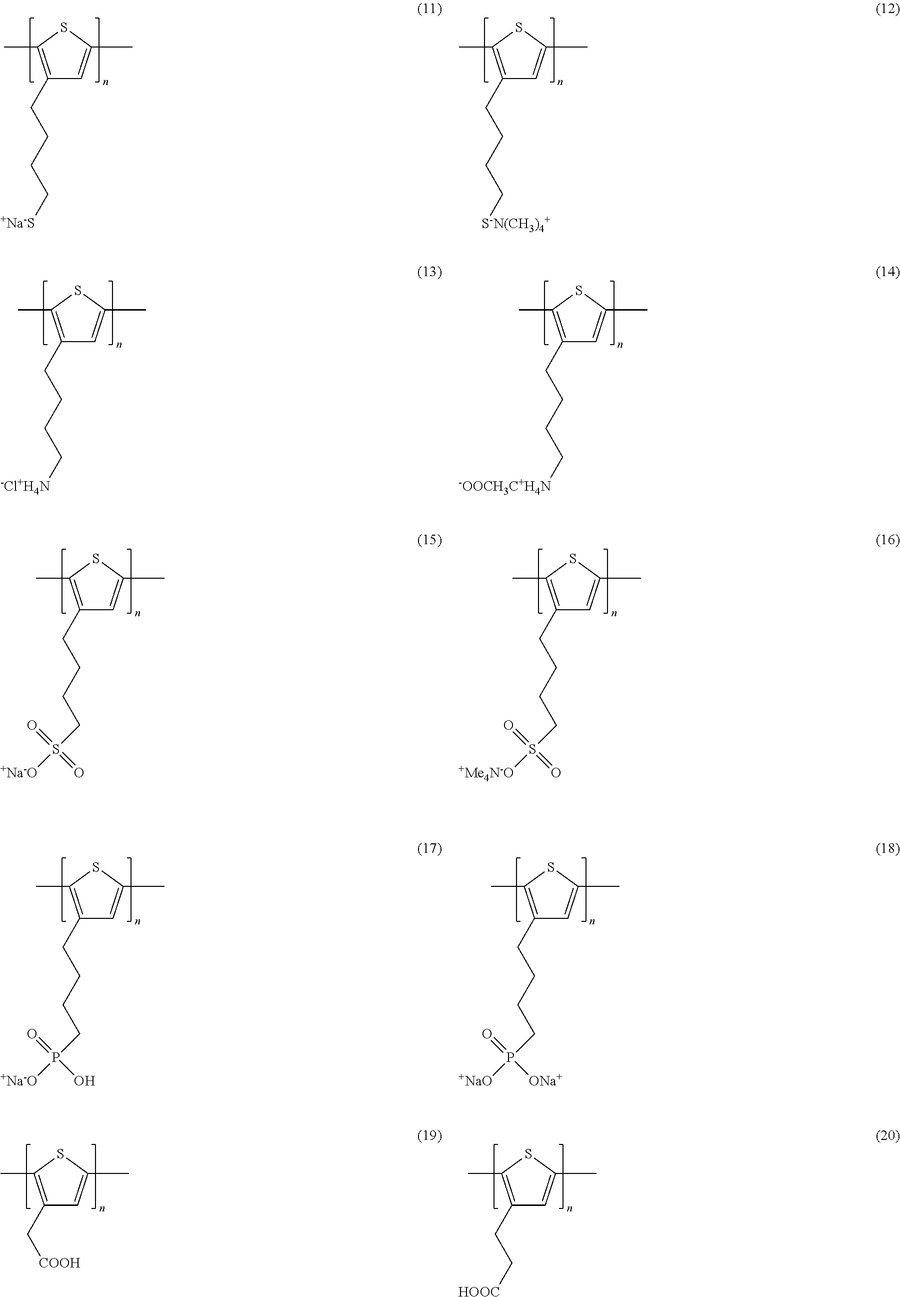

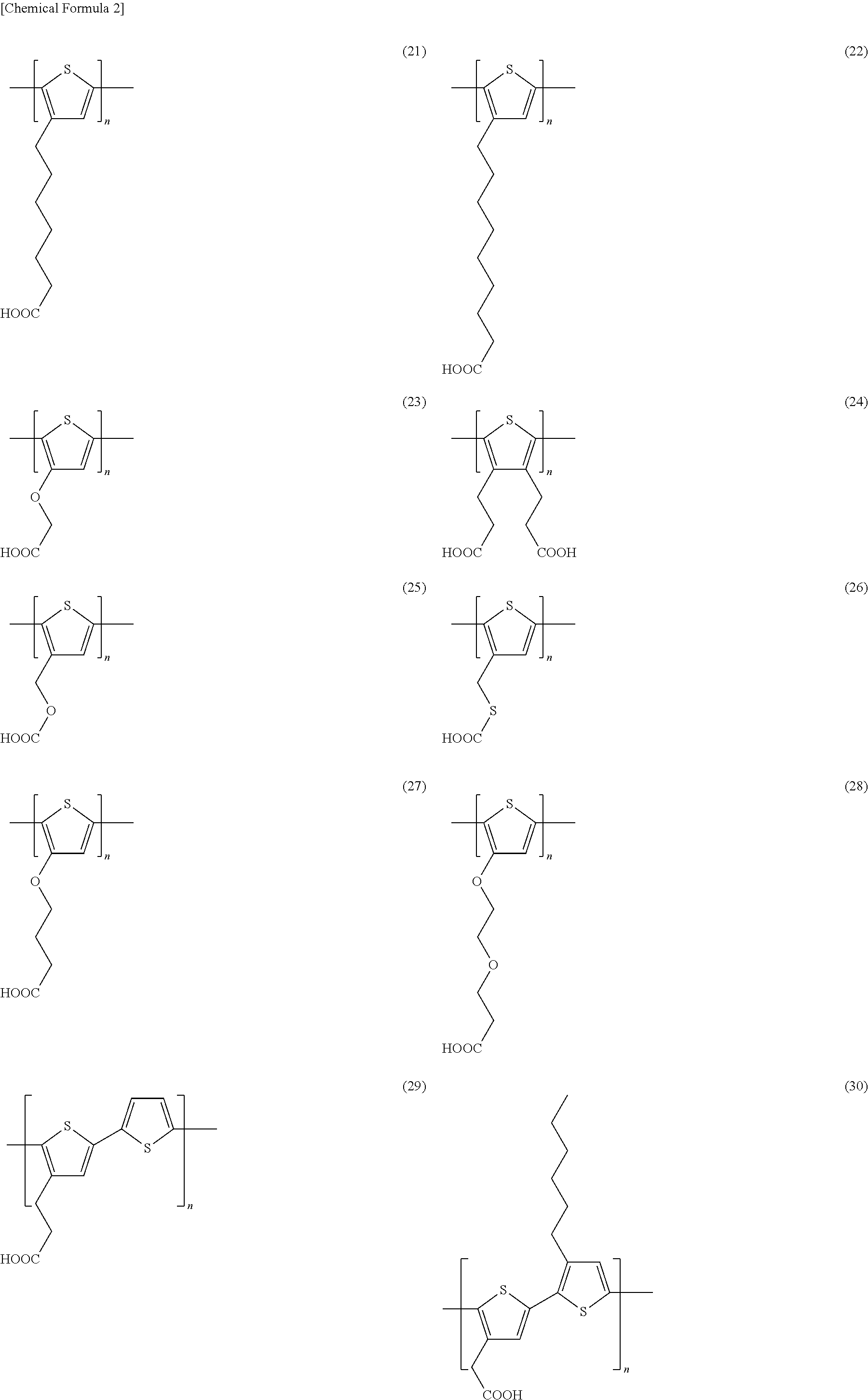

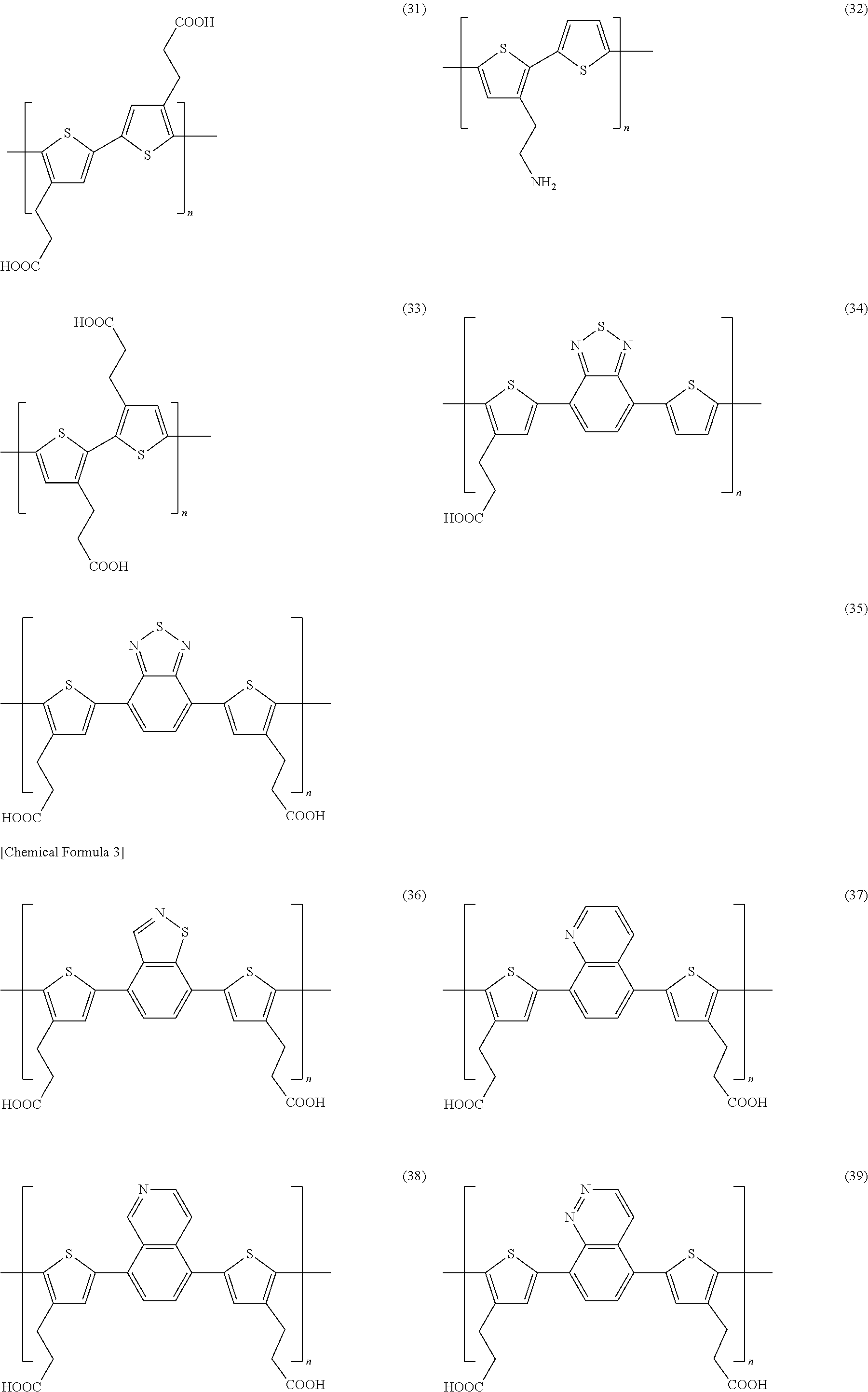

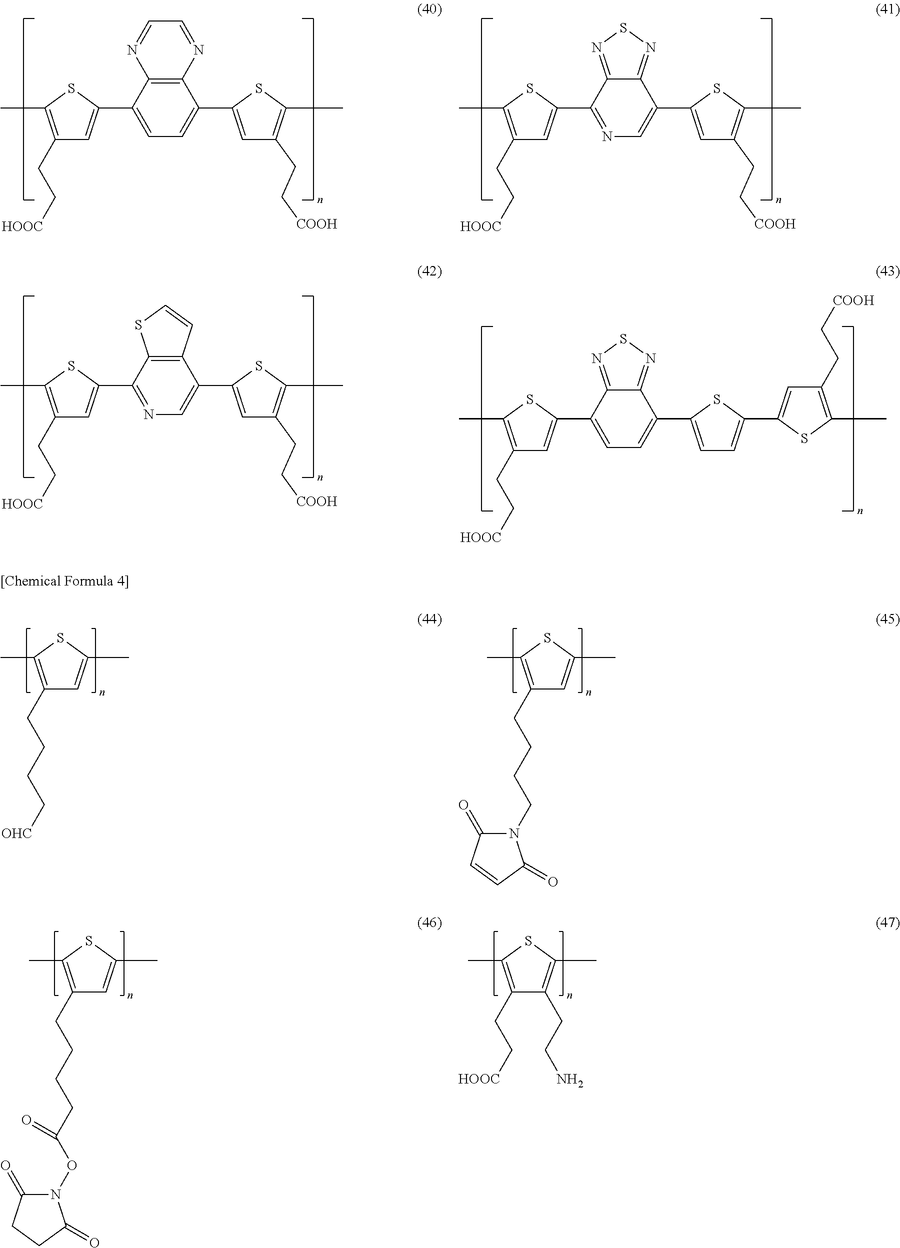

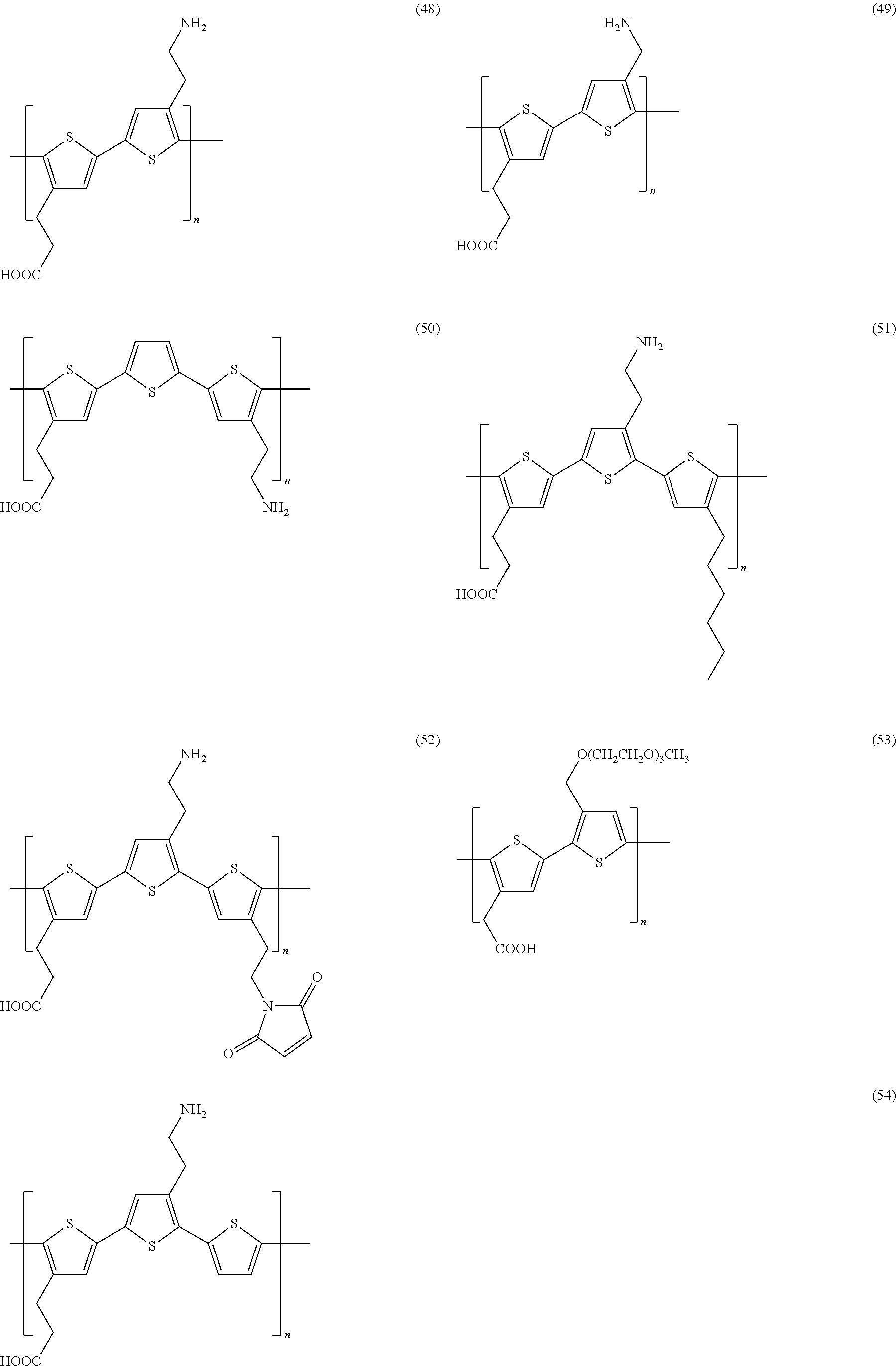

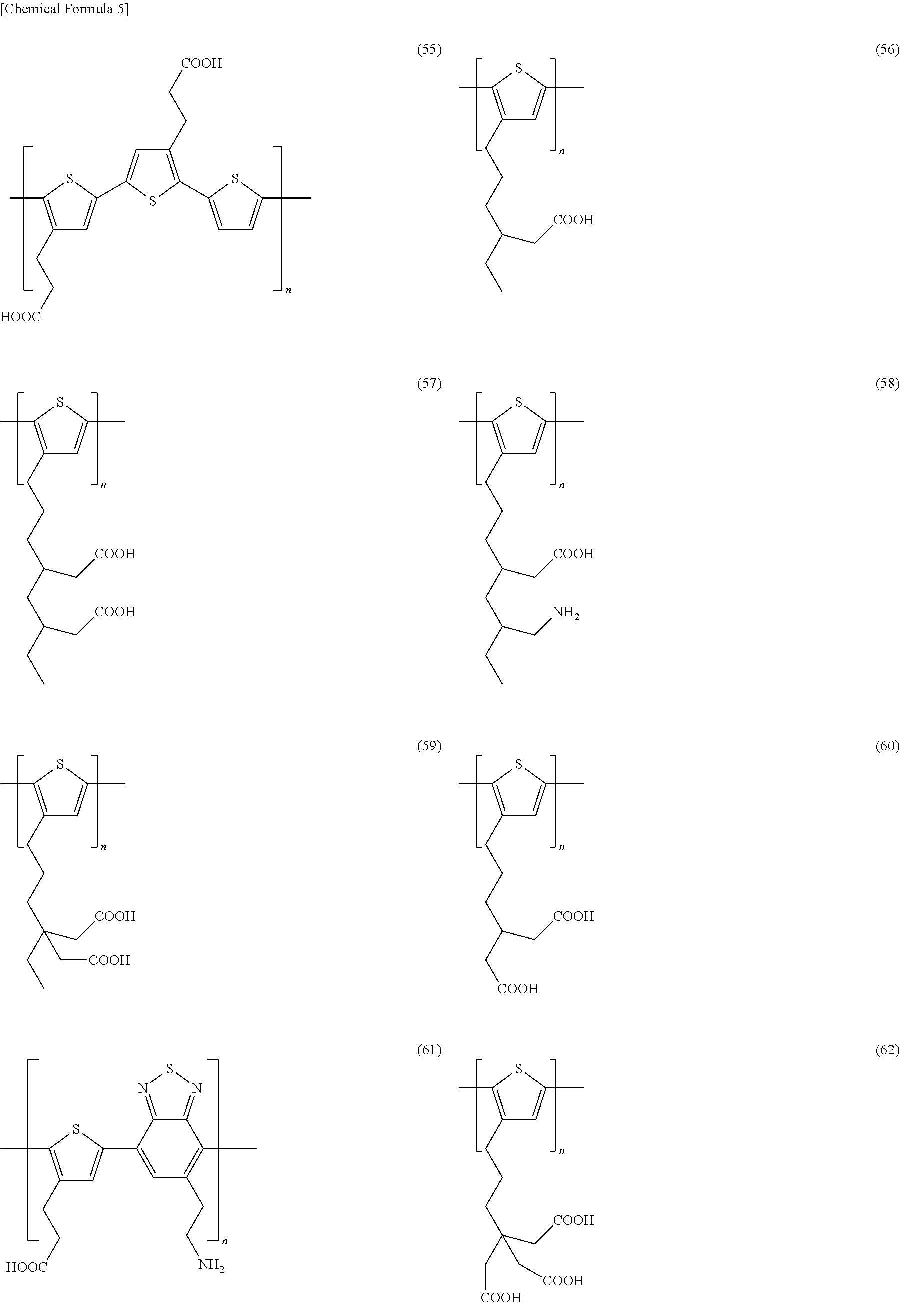

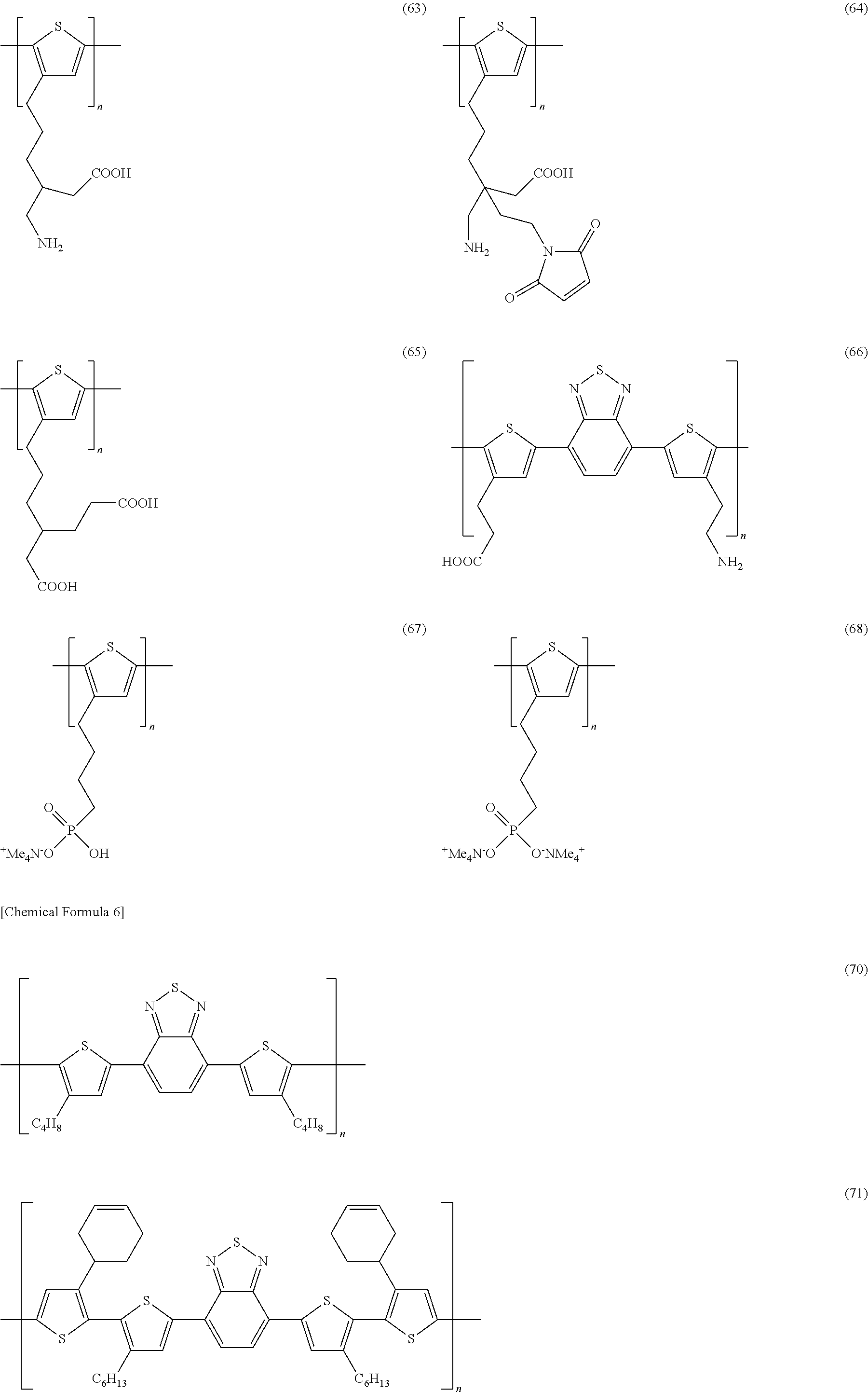

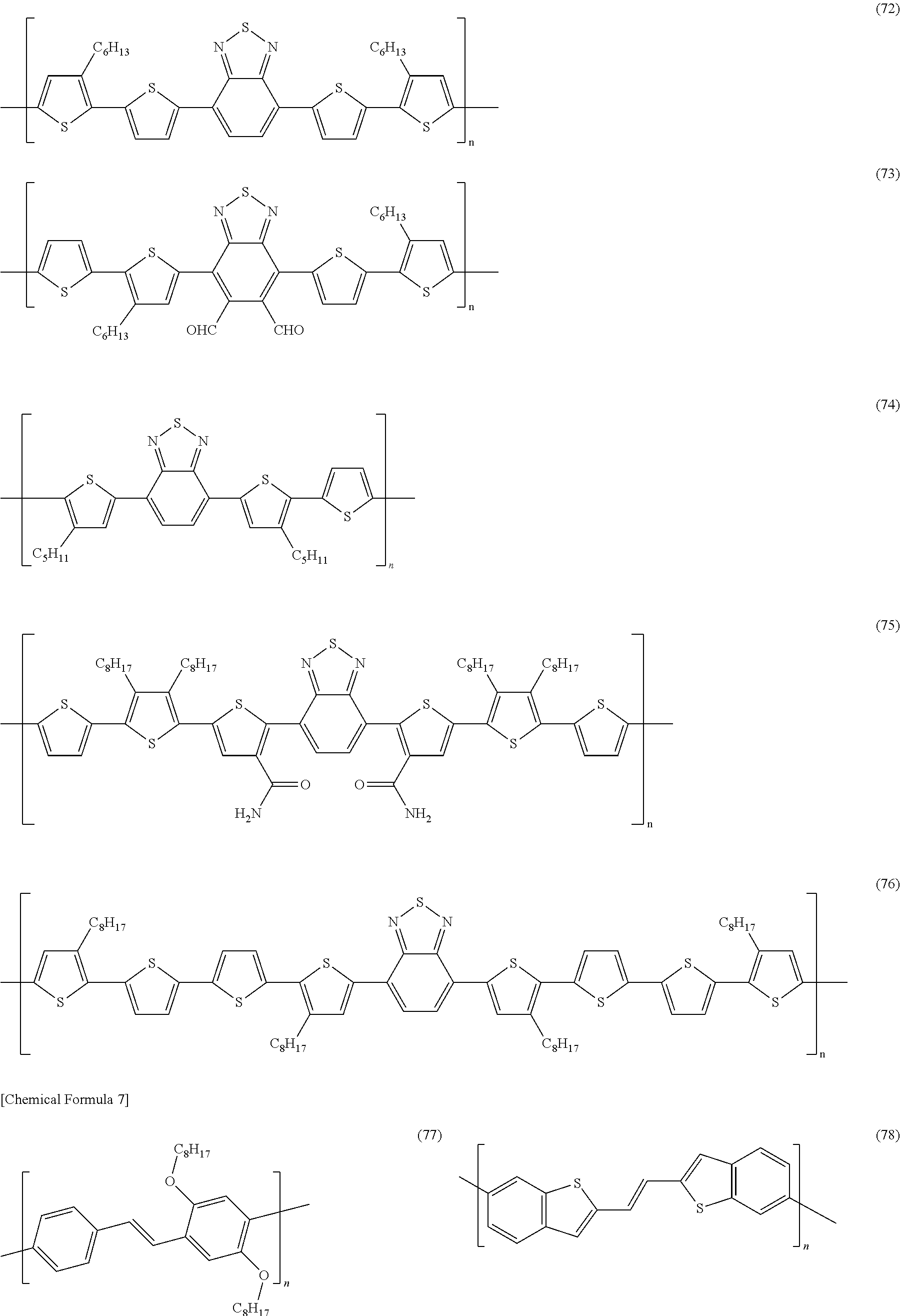

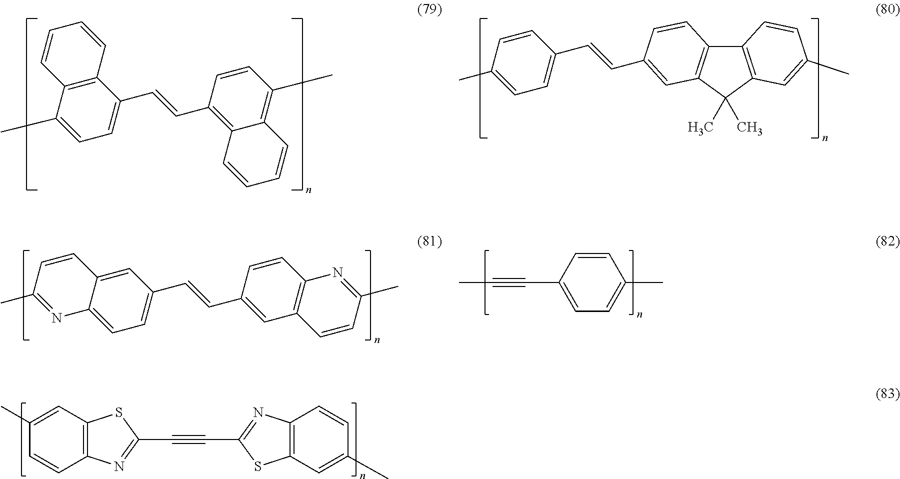

The conjugated polymer used in the present invention can be synthesized by a known method. For the synthesis of a monomer, for example, methods that may be used for coupling a thiophene derivative having introduced thereinto side chains to thiophene include: a method in which a halogenated thiophene derivative and thiopheneboronic acid or a thiopheneboronic acid ester are coupled in the presence of a palladium catalyst; and a method in which a halogenated thiophene derivative and a thiophene Grignard reagent are coupled in the presence of a nickel or palladium catalyst. In addition, also in the case of coupling a unit other than the above thiophene derivative to thiophene, coupling can be performed in the same manner using a halogenated unit. In addition, it is possible that polymerizable substituents are introduced into the terminus of the monomer thus obtained, and polymerization is allowed to proceed in the presence of a palladium catalyst or a nickel catalyst, thereby giving a conjugated polymer.

With respect to the conjugated polymer used in the present invention, it is preferable that impurities, such as raw materials used in the course of synthesis and by-products, are removed. For example, a silica gel column chromatography method, a Soxhlet extraction method, a filtration method, an ion-exchange method, a chelating method, and the like may be used. It is also possible to combine two or more of these methods.

(Blocking Agent (B))

A blocking agent is a compound that is attached to the surface of CNTs and thus has the effect of preventing the adsorption of non-target proteins onto the surface of CNTs.

The blocking agent is not particularly limited. More specifically, compounds that may be used as the blocking agent include polyvinyl alcohol, celluloses such as carboxymethylcellulose, polyalkylene glycols such as polyethylene glycol, acrylic resins such as polyhydroxymethyl methacrylate, phospholipids such as phosphatidylcholine, and proteins such as bovine serum albumin (BSA). In terms of the adsorption-preventing effect, it is preferable that the blocking agent is selected from: a compound having at least one of a tetraalkylammonium structure and a phosphoester structure as a partial structure (B1); a polysaccharide (B2); an albumin (B3); and a phospholipid (B4).

Examples of compounds (B1) include compounds having a tetraalkylammonium structure as a partial structure, such as hexadecyltrimethylammonium bromide, stearyltrimethylammonium bromide, and ethyl sulfate lanolin fatty acid amino propyl ethyl dimethyl ammonium, and compounds having a phosphoester structure as a partial structure, such as sodium laurylphosphate, riboflavin sodium phosphate, and adenosine triphosphate.

Examples of polysaccharides (B2) include amylose, cellulose, and carboxymethylcellulose.

Examples of albumins (B3) include human serum albumin, bovine serum albumin, rabbit serum albumin, and ovalbumin.

Examples of phospholipids (B4) include phosphatidic acid, phosphatidylcholine, phosphatidylethanolamine, phosphatidylserine, phosphatidylinositol, and sphingomyelin.

In terms of the interaction with CNTs, phospholipids and serum albumins are more preferable, and bovine serum albumin is particularly preferable.

It is preferable that the thickness of the blocking agent is 50 nm or less. When the thickness is within this range, in the case where the CNT composite of the present invention is applied to a sensor, changes in electrical characteristics caused by the interaction with a substance to be sensed can be sufficiently taken out as an electrical signal. The thickness is more preferably 30 nm or less, and still more preferably 10 nm or less. The lower limit of the thickness of the blocking agent is not particularly limited, but is preferably 1 nm or more. The thickness of the blocking agent can be measured using an atomic force microscope.

(Functional Group)

It is preferable that at least a portion of the CNT composite of the present invention has at least one functional group selected from the group consisting of a hydroxyl group, a carboxy group, an amino group, a mercapto group, a sulfo group, a phosphonic acid group, an organic salt or inorganic salt thereof, a formyl group, a maleimide group, and a succinimide group. As a result, it becomes easier to detect a substance to be sensed. More specifically, these functional groups undergo an interaction with a substance to be sensed, such as chemical bonding, hydrogen bonding, ionic bonding, coordinate bonding, electrostatic interaction, or oxidation-reduction reaction. This results in changes in the electrical characteristics of CNTs that are present in the vicinity, and such changes can be more easily detected as an electrical signal.

Among the functional groups described above, an amino group, a maleimide group, and a succinimide group may have a substituent. Examples of substituents include an alkyl group, and such substituents may further be substituted.

In the functional groups described above, organic salts are not particularly limited, and examples thereof include ammonium salts such as a tetramethylammonium salt, pyridinium salts such as an N-methylpyridinium salt, imidazolium salts, carboxylates such as an acetate, sulfonates, and phosphonates.

In the functional groups described above, inorganic salts are not particularly limited, and examples thereof include carbonates, alkali metal salts such as a sodium salt, alkaline earth metal salts such as a magnesium salt, salts of transition metal ions such as copper, zinc, and iron, salts of boron compounds such as tetrafluoroborate, sulfates, phosphates, hydrochlorides, and nitrates.

Modes of the introduction of a functional group into the CNT composite include: a mode in which a portion of the aggregation inhibitor or blocking agent attached to the surface of CNTs has a functional group; and a mode in which an organic compound (C) different from the aggregation inhibitor and the blocking agent is attached to the surface of CNTs, and a portion of the organic compound has a functional group. In terms of detection sensitivity, a mode in which an organic compound (C) different from the aggregation inhibitor and the blocking agent is attached to the surface of CNTs, and a portion of the organic compound has a functional group, is more preferable.

Examples of organic compounds (C) having a functional group include stearylamine, laurylamine, hexylamine, 1, 6-diaminohexane, diethylene glycol bis (3-aminopropyl) ether, isophoronediamine, 2-ethylhexylamine, stearic acid, lauric acid, sodium dodecyl sulfate, Tween20, 1-pyrenecarboxylic acid, 1-aminopyrene, 1-hexabenzocoronenecarboxylic acid, 1-aminohexabenzocoronene, 1-hexabenzocoronenebutanecarboxylic acid, 1-pyrenebutanecarboxylic acid, 4-(pyren-1-yl)butan-1-amine, 4-(pyren-1-yl)butan-1-ol, 4-(pyren-1-yl)butane-1-thiol, 4-(hexabenzocoronen-1-yl)butan-1-amine, 4-(hexabenzocoronen-1-yl)butan-1-ol, 4-(hexabenzocoronen-1-yl)butane-1-thiol, 1-pyrenebutanecarboxylic acid-N-hydroxysuccinimide ester, 1-hexabenzocoronenebutanecarboxylic acid-N-hydroxysuccinimide ester, biotin, biotin-N-hydroxysuccinimide ester, biotin-N-hydroxy-sulfosuccinimide ester, polyethyleneimine, polyethylene glycol, polyvinyl alcohol, polyacrylic acid, sodium polyacrylate, polyacrylamine, polyacrylamine hydrochloride, polymethacrylic acid, sodium polymethacrylate, polymethacrylamine, polymethacrylamine hydrochloride, alginic acid, sodium alginate, glucose, maltose, sucrose, chitin, amylose, amylopectin, cellulose, carboxymethylcellulose, sucrose, lactose, cholic acid, sodium cholate, deoxycholic acid, sodium deoxycholate, cholesterol, cyclodextrin, xylan, catechin, poly-3-(ethylsulfonic acid-2-yl)thiophene, poly-3-(ethanoic acid-2-yl)thiophene, poly-3-(2-aminoethyl)thiophene, poly-3-(2-hydroxyethyl)thiophene, poly-3-(2-mercaptoethyl)thiophene, polystyrene sulfonic acid, polyvinylphenol, polyoxypropylene triol, glutaraldehyde, ethylene glycol, ethylenediamine, poly-1H-(propionic acid-3-yl)pyrrole, 1-adamantanol, 2-adamantanol, 1-adamantanecarboxylic acid, dodecylbenzenesulfonic acid, sodium dodecylbenzenesulfonate, and N-ethylmaleimide. The above organic compounds may be used alone, and it is also possible to use two or more kinds of organic compounds.

Methods for attaching an organic compound (C) to CNTs include:

(I) a method in which CNTs are added to a melted organic compound and mixed;

(II) a method in which the organic compound is dissolved in a solvent, and CNTs are added thereto and mixed;

(III) a method in which CNTs are pre-dispersed ultrasonically, etc., and the organic compound is added thereto and mixed;

(IV) a method in which the organic compound and CNTs are added to a solvent, and the mixed system is mixed by ultrasonic irradiation;

(V) a method in which CNTs applied onto a substrate are immersed in a melted organic compound; and

(VI) a method in the organic compound is dissolved in a solvent, and CNTs applied onto a substrate are immersed therein.

In the present invention, any of these methods may be used, and it is also possible to use any combination of these methods.

The order of attaching an aggregation inhibitor, a blocking agent, and an organic compound (C) to CNTs is not particularly limited. However, it is preferable that (1) the aggregation inhibitor is attached, then the organic compound is attached, and subsequently the blocking agent is attached, or (2) the aggregation inhibitor and the organic compound are attached at the same time, and then the blocking agent is attached.

(Bio-Related Material)

In the CNT composite of the present invention, it is preferable that a bio-related material that selectively interacts with a substance to be sensed is immobilized on at least a portion of a surface. As a result, a substance to be sensed can be selectively immobilized on the CNT composite surface.

The bio-related material is not particularly limited as long as it can selectively interact with a substance to be sensed, and arbitrary substances may be used. Specific examples thereof include enzymes, antigens, antibodies, haptens, hapten antibodies, peptides, oligopeptides, polypeptides (proteins), hormones, nucleic acids, oligonucleotides, biotin, biotinylated proteins, avidin, streptavidin, saccharides such as sugar, oligosaccharides, and polysaccharides, low-molecular-weight compounds, high-molecular-weight compounds, inorganic substances, composites thereof, viruses, bacteria, cells, biological tissues, and substances constituting them. Among them, biotin and IgE aptamer are more preferable.

The state in which a bio-related material is immobilized on at least a portion of the surface of the CNT composite means the state in which the bio-related material is adsorbed or bound to the surface of the CNT composite.

The method for immobilizing a bio-related material to the surface of the CNT composite is not particularly limited, and examples of the method include: (1) a method in which the bio-related material is directly adsorbed to the CNT composite surface; and (2) a method that utilizes the reaction or interaction between the bio-related material and the functional group contained in the CNT composite, that is, at least one functional group selected from the group consisting of a hydroxyl group, a carboxy group, an amino group, a mercapto group, a sulfo group, a phosphonic acid group, an organic salt or inorganic salt thereof, a formyl group, a maleimide group, and a succinimide group. In terms of the strength of immobilization, it is preferable to utilize the reaction or interaction between the bio-related material and the functional group contained in the CNT composite (2). For example, in the case where an amino group is contained in the bio-related material, a carboxy group, an aldehyde group, and a succinimide group are preferable. In the case of a thiol group, a maleimide group and the like are preferable.

Among the above groups, a carboxy group and an amino group facilitate the utilization of the reaction or interaction with the bio-related material, and the bio-related material can be easily immobilized on a semiconductor layer. Therefore, it is preferable that the functional group contained in at least a portion of the CNT composite is a carboxy group, a succinimide ester group, or an amino group.

Specific examples of reactions or interactions include, but are not particularly limited to, chemical bonding, hydrogen bonding, ionic bonding, coordinate bonding, electrostatic force, and van der Waals force. Suitable selection should be made according to the kind of functional group and the chemical structure of the bio-related material. In addition, as necessary, immobilization may be performed after converting a portion of the functional group and/or bio-related material into a different, suitable functional group.

In addition, it is also possible to use a linker, such as terephthalic acid, between the functional group and the bio-related material.

The immobilizing process is not particularly limited and may be a process in which, for example, a solution containing the bio-related material is added to a solution or substrate containing the CNT composite, then the bio-related material is immobilized with heating, cooling, vibration, or the like as necessary, and subsequently excess components are removed by washing or drying.

In the CNT composite of the present invention, examples of combinations of functional group contained in the CNT composite/bio-related material include carboxy group/glucose oxidase, carboxy group/T-PSA-mAb (monoclonal antibody for prostate-specific antigen), carboxy group/hCG-mAb (anti-human chorionic gonadotropin), carboxy group/artificial oligonucleotide (IgE (immunoglobulin E) aptamer), carboxy group/IgE, carboxy group/amino group-terminated RNA (HIV-1 (human immunodeficiency virus) receptor), carboxy group/natriuretic peptide receptor, amino group/RNA (HIV-1 antibody receptor), amino group/biotin, mercapto group/T-PSA-mAb, mercapto group/hCG-mAb, sulfo group/T-PSA-mAb, sulfo group/hCG-mAb, phosphonic acid group/T-PSA-mAb, phosphonic acid group/hCG-mAb, aldehyde group/oligonucleotide, aldehyde group/anti-AFP polyclonal antibody (antibody for immunostaining of human tissue), maleimide group/cysteine, succinimide ester/streptavidin, sodium carboxylate/glucose oxidase, carboxy group/anti-troponin T (troponin T antibody), carboxy group/anti-CK-MB (creatinine kinase MB antibody), carboxy group/anti-PIVKA-II (protein induced by vitamin K absence or antagonist-II antibody), carboxy group/anti-CA15-3, carboxy group/anti-CEA (carcinoembryonic antigen antibody), carboxy group/anti-CYFRA (cytokeratin 19 fragment antibody), and carboxy group/anti-p53 (p53 protein antibody). In addition, in the case where the bio-related material contains a functional group, such a bio-related material may be preferably used as an organic compound containing a functional group. Specific examples thereof include IgE aptamer, biotin, streptavidin, natriuretic peptide receptor, avidin, T-PSA-mAb, hCG-mAb, IgE, amino group-terminated RNA, RNA, anti-AFP polyclonal antibody, cysteine, anti-troponin T, anti-CK-MB, anti-PIVKA-II, anti-CA15-3, anti-CEA, anti-CYFRA, and anti-p53.

<Semiconductor Device>

The semiconductor device of an embodiment of the present invention includes a substrate, a first electrode, a second electrode, and a semiconductor layer, wherein the first electrode is spaced apart from the second electrode, the semiconductor layer is disposed between the first electrode and the second electrode, and the semiconductor layer contains the CNT composite of the present invention. In addition, according to another aspect, the semiconductor device further includes a gate electrode and an insulating layer, and the gate electrode is electrically insulated from the first electrode, the second electrode, and the semiconductor layer by the insulating layer.

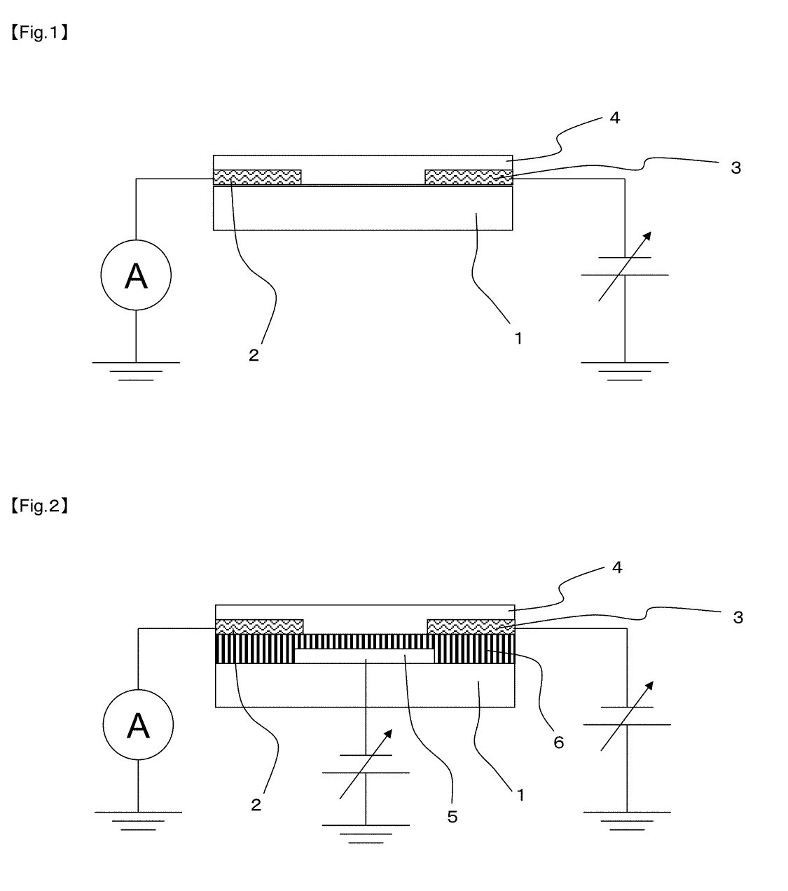

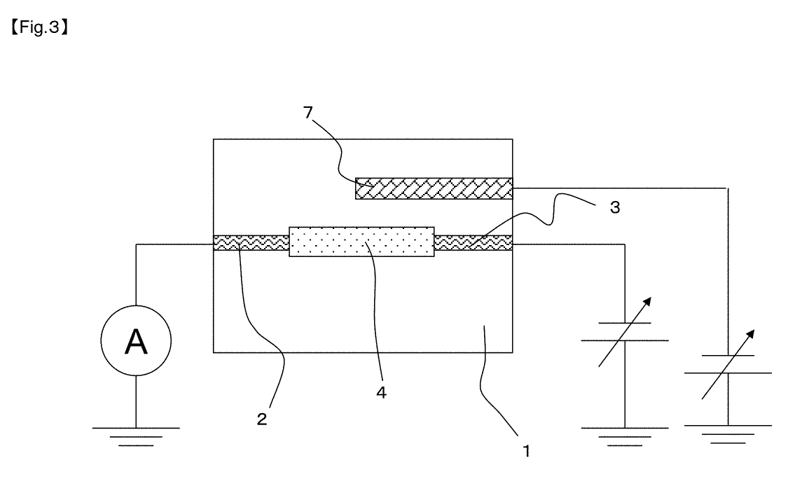

FIG. 1 and FIG. 2 are schematic cross-sectional views each showing an example of the semiconductor device of the present invention. The semiconductor device of FIG. 1 is configured such that a first electrode 2 and a second electrode 3 are formed on a substrate 1, and a semiconductor layer 4 is disposed between the first electrode 2 and the second electrode 3. The semiconductor device of FIG. 2 is configured such that a gate electrode 5 and an insulating layer 6 are formed on a substrate 1, a first electrode 2 and a second electrode 3 are formed thereon, and a semiconductor layer 4 containing the CNT composite of the present invention is disposed between the first electrode 2 and the second electrode 3. In the semiconductor device of FIG. 2, the first electrode 2 and the second electrode 3 correspond to a source electrode and a drain electrode, respectively, and the insulating layer 6 corresponds to a gate insulating layer, whereby the semiconductor device functions as an FET.

Examples of materials used for the substrate 1 include inorganic materials, such as silicon wafers, glass, and alumina sintered bodies, and organic materials, such as polyimide, polyester, polycarbonate, polysulfone, polyethersulfone, polyethylene, polyphenylene sulfide, and polyparaxylene.

Examples of materials used for the first electrode 2, the second electrode 3, and the gate electrode 5 include, but are not limited to, electrically conductive metal oxides such as tin oxide, indium oxide, and tin oxide indium (ITO), metals such as platinum, gold, silver, copper, iron, tin, zinc, aluminum, indium, chromium, lithium, sodium, potassium, cesium, calcium, magnesium, palladium, molybdenum, amorphous silicon, and polysilicon, as well as alloys thereof, inorganic electrically conductive substances such as copper iodide and copper sulfide, organic electrically conductive substances such as polythiophene, polypyrrole, polyaniline, and a polyethylene dioxythiophene-polystyrene sulfonic acid complex, and nano-carbon materials such as carbon nanotubes and graphene. These electrode materials may be used alone, and it is also possible to laminate or mix and a plurality of materials.

In a sensor application, in terms of stability in an aqueous solution or the like that the sensor contacts, it is preferable that the first electrode 2 and the second electrode 3 are selected from gold, platinum, palladium, organic electrically conductive substances, and nano-carbon materials.

With respect to the first electrode, the second electrode, and the gate electrode, the width, thickness, spacing, and disposition are arbitrary. It is preferable that the width is 1 μm to 1 mm, the thickness is 1 nm to 1 μm, and the electrode spacing is 1 μm to 10 mm. For example, electrodes 100 μm wide and 500 nm thick are disposed as a first electrode and a second electrode with a space of 2 mm, and a gate electrode 100 μm wide and 500 nm thick is disposed therebelow, but the disposition is not limited thereto.

Examples of materials used for the insulating layer 6 include inorganic materials such as silicon oxide and alumina, organic polymer materials such as polyimide, polyvinyl alcohol, polyvinyl chloride, polyethylene terephthalate, polyvinylidene fluoride, polysiloxane, and polyvinylphenol (PVP), and mixtures of an inorganic material powder and an organic polymer material.

It is preferable that the thickness of the insulating layer 6 is 10 nm or more and 5 μm or less. The thickness is more preferably 50 nm or more and 3 μm or less, and still more preferably 100 nm or more and 1 μm or less. The thickness can be measured by an atomic force microscope, ellipsometry, or the like.

The semiconductor layer 4 contains the CNT composite of the present invention. Without inhibiting the electrical characteristics of the CNT composite, the semiconductor layer 4 may further contain an organic semiconductor or an insulating material.

It is preferable that the thickness of the semiconductor layer 4 is 1 nm or more and 100 nm or less. When the thickness is within this range, changes in electrical characteristics caused by the interaction with a substance to be sensed can be sufficiently taken out as an electrical signal. The thickness is more preferably 1 nm or more and 50 nm or less, and still more preferably 1 nm or more and 20 nm or less.

In the semiconductor layer 4, in terms of detection sensitivity, it is preferable that a functional group is contained only in the vicinity of the CNT composite, and it is particularly preferable that a functional group is contained only in the surface of the CNT composite. Particularly in the case where the semiconductor layer contains an organic compound (C), it is preferable that 70 wt % or more of the organic compound (C) present on the semiconductor device surface is attached to the surface of CNTs.

As a method for forming the semiconductor layer 4, although it is possible to use a dry method such as resistance heating vapor deposition, electron beam, sputtering, or CVD, in terms of production cost and adaptation to a large area, it is preferable to use a coating method. Specifically, a spin coating method, a blade coating method, a slit die coating method, a screen printing method, a bar coater method, a template method, a printing transfer method, a dipping pulling-up method, an ink-jet method, and the like may be preferably used. The coating method may be selected according to the coating film characteristics to be obtained, including coating film thickness control, orientation control, and the like. In addition, the formed coating film may also be subjected to an annealing treatment in air, vacuum, or an inert gas atmosphere (in a nitrogen or argon atmosphere).

The semiconductor layer 4 can be formed by applying a solution containing the CNT composite of the present invention. The solvent is not particularly limited, and examples thereof include water, ethanol, tetrahydrofuran, acetonitrile, N-methylpyrrolidone, γ-butyrolactone, propyleneglycol-1-monomethyl ether 2-acetate, chloroform, o-dichlorobenzene, and toluene. The above solvents may be used alone, and it is also possible to use a mixture of two or more kinds of solvents. The solvent is suitably selected and used according to the kinds of aggregation inhibitor, blocking agent, and functional group.

In the semiconductor layer 4, surface protection and the immobilization of a bio-related material are not particularly limited. However, in terms of detection sensitivity, it is preferable that a CNT composite wherein an aggregation inhibitor is attached to at least a portion of the surface of CNTs is applied onto a substrate, and then a blocking agent is attached to the CNT composite. It is also preferable that a bio-related material that selectively interacts with a substance to be sensed is immobilized on the CNT composite. The method for surface protection is as described above. As necessary, excess components may be removed by washing or drying.

The immobilization of a bio-related material is not particularly limited. However, the following methods are preferable: (1) in terms of detection sensitivity, a method in which a CNT composite wherein an aggregation inhibitor is attached to at least a portion of the surface of CNTs is applied onto a substrate, then a blocking agent is attached to the CNT composite, and further a bio-related material that selectively interacts with a substance to be sensed is immobilized on the CNT composite by the above method, and (2) a method in which a carbon nanotube composite wherein an aggregation inhibitor is attached to at least a portion of the surface of CNTs is applied onto a substrate, then a bio-related material that selectively interacts with a substance to be sensed is immobilized on the CNT composite by the above method, and further a blocking agent is attached to the CNT composite. A specific example of the method for immobilizing a bio-related material is a method in which a bio-related material is dissolved in a solvent, and the above substrate is immersed in the solution. As necessary, excess components may be removed by washing or drying.

In an FET, the current flowing between the source electrode and the drain electrode can be controlled by changing the gate voltage. The mobility of an FET can be calculated using the following equation (a).

μ=(δId/δVg)L·D/(W·εᵣ·ε·Vsd) (a)

wherein Id is the current between the source and drain, Vsd is the voltage between the source and drain, Vg is the gate voltage, D is the thickness of the insulating layer, L is the channel length, W is the channel width, εᵣ is the relative dielectric constant of the gate insulating layer, and ε is the dielectric constant of vacuum (8.85×10⁻¹² F/m).

In addition, the on-off ratio can be determined from the ratio between the maximum Id and the minimum Id.

<Sensor>

The sensor of the present invention includes the semiconductor device described above. That is, the sensor includes a semiconductor device including a substrate, a first electrode, a second electrode, and a semiconductor layer, wherein the first electrode is spaced apart from the second electrode, the semiconductor layer is disposed between the first electrode and the second electrode, and the semiconductor layer contains the carbon nanotube composite according to any one of claims 1 to 6. Further, it is preferable that the sensor of the present invention contains, in the semiconductor layer, a bio-related material that selectively interacts with a substance to be sensed.

In the sensor including a semiconductor device formed as shown in FIG. 1, when a substance to be sensed or a solution, gas, or solid containing the substance is disposed in the vicinity of the semiconductor layer 4, the value of the current flowing between the first electrode and the second electrode or the electric resistance value changes. By measuring such changes, the substance to be sensed can be detected.

In addition, also in the sensor including a semiconductor device formed as shown in FIG. 2, when a substance to be sensed or a solution, gas, or solid containing the substance is disposed in the vicinity of the semiconductor layer 4, the value of the current flowing between the first electrode 2 and the second electrodes 3, that is, flowing through the semiconductor layer 4, changes. By measuring such changes, the substance to be sensed can be detected.

In addition, in the sensor including the semiconductor device of FIG. 2, the value of the current flowing through the semiconductor layer 4 can be controlled by the voltage on the gate electrode 5. Accordingly, by measuring the value of the current flowing between the first electrode 2 and the second electrode 3 upon a voltage change in the gate electrode 5, a two-dimensional graph (I-V graph) is obtained.

A substance to be sensed may be detected using some or all of such characteristic values. Alternatively, the detection of a substance to be sensed may also be performed using the ratio between the maximum current and the minimum current, that is, the on-off ratio. Further, it is also possible to use known electrical characteristics obtained from the semiconductor device, such as resistance, impedance, transconductance, and capacitance.

The substance to be sensed may be used alone, and may also be mixed with other substances or solvents. The substance to be sensed or a solution, gas, or solid containing the substance is disposed in the vicinity of the semiconductor layer 4. As described above, when the semiconductor layer 4 interacts with the substance to be sensed, the electrical characteristics of the semiconductor layer 4 change, and such changes are detected as any of the above electrical signals.

In addition, in the sensor of the present invention, when the surface of CNTs is protected with a blocking agent, the detection of non-target proteins can be prevented, and the substance to be sensed can be selectively detected.

The substance to be sensed by the sensor of the present invention is not particularly limited, and examples thereof include enzymes, antigens, antibodies, haptens, peptides, oligopeptides, polypeptides (proteins), hormones, nucleic acids, oligonucleotides, saccharides such as sugar, oligosaccharides, and polysaccharides, low-molecular-weight compounds, inorganic substances, composites thereof, viruses, bacteria, cells, biological tissues, and substances constituting them. They react or interact with at least one member selected from the group consisting of a hydroxyl group, a carboxy group, an amino group, a mercapto group, a sulfo group, a phosphonic acid group, an organic salt or inorganic salt thereof, a formyl group, a maleimide group, and a succinimide group, or with a bio-related material, and such a reaction or interaction results in changes in the electrical characteristics of the semiconductor layer in the sensor of the present invention.

The low-molecular-weight compound is not particularly limited, and examples thereof include compounds that are gases at normal temperature and normal pressure, such as ammonia and methane emitted from the living body, and solid compounds, such as uric acid.

In the sensor of the present invention, examples of combinations of bio-related material/substance to be sensed include glucose oxidase/(β-D-glucose, T-PSA-mAb (monoclonal antibody for prostate-specific antigen)/PSA (prostate-specific antigen), hCG-mAb(anti-human chorionic gonadotropin)/hCG (human chorionic gonadotropin), artificial oligonucleotide/IgE (immunoglobulin E), diisopropylcarbodiimide/IgE, amino group-terminated RNA/HIV-1 (human immunodeficiency virus), natriuretic peptide receptor/BNP (brain natriuretic peptide), RNA/HIV-1, biotin/avidin, oligonucleotide/nucleic acid, anti-AFP polyclonal antibody (antibody for immunostaining of human tissue)/a-fetoprotein, streptavidin/biotin, anti-troponin T (troponin T antibody)/troponin T, anti-CK-MB (creatinine kinase MB antibody)/CK-MB (creatinine kinase MB), anti-PIVKA-II (protein induced by vitamin K absence or antagonist-II antibody)/PIVKA-II (protein induced by vitamin K absence or antagonist-II), anti-CA15-3/CA15-3, anti-CEA (carcinoembryonic antigen antibody)/CEA (carcinoembryonic antigen), anti-CYFRA (cytokeratin 19 fragment antibody)/CYFRA (cytokeratin 19 fragment), and anti-p53 (p53 protein antibody)/p53 (p53 protein).

It is preferable that the sensor of the present invention further includes a third electrode. That is, it is preferable that the sensor includes a semiconductor device including a substrate, a first electrode, a second electrode, a third electrode, and a semiconductor layer, wherein the first electrode is spaced apart from the second electrode, the semiconductor layer is disposed between the first electrode and the second electrode, and the semiconductor layer contains the CNT composite of the present invention. As a result, changes in the electrical characteristics of the semiconductor layer are caused by voltage applying to the semiconductor layer through the third electrode, whereby the detection sensitivity can be improved.

FIG. 3 is a schematic plan view showing an example of the sensor of the present invention. In the sensor of FIG. 3, a first electrode 2 and a second electrode 3 are formed on a substrate 1, a semiconductor layer 4 is disposed between the first electrode 2 and the second electrode 3, and a third electrode 7 is further disposed on the substrate 1.

With respect to the third electrode, the width, thickness, distance from the semiconductor layer, and disposition are arbitrary. It is preferable that the width is 1 μm to 1 mm, the thickness is 1 nm to 1 μm, and the distance from the semiconductor layer is 1 μm to 10 cm. For example, an electrode 100 μm wide and 500 nm thick is disposed at a distance of 2 mm from the semiconductor layer, but the disposition isnot limited thereto. In FIG. 3, the third electrode 7 is disposed parallel to the second electrode 3, but it may also be disposed perpendicularly or at another arbitrary angle. The shape of the third electrode 7 is not limited to a straight line and may also be a curved line. The third electrode 7 does not have to be disposed right above the substrate 1 and may also be disposed on a different member disposed on the substrate 1.

Examples of materials used for the third electrode 7 include, but are not limited to, electrically conductive metal oxides such as tin oxide, indium oxide, and tin oxide indium (ITO), metals such as platinum, gold, silver, copper, iron, tin, zinc, aluminum, indium, chromium, lithium, sodium, potassium, cesium, calcium, magnesium, palladium, molybdenum, amorphous silicon, and polysilicon, as well as alloys thereof, inorganic electrically conductive substances such as copper iodide, copper sulfide, and silver-silver chloride, organic electrically conductive substances such as polythiophene, polypyrrole, polyaniline, and a polyethylene dioxythiophene-polystyrene sulfonic acid complex, and nano-carbon materials such as carbon nanotubes and graphene. These electrode materials may be used alone, and it is also possible to laminate or mix and a plurality of materials. In a sensor application, in terms of stability in an aqueous solution or the like that the sensor contacts, it is preferable that the first electrode 2, the second electrode 3, and the third electrode 7 are selected from gold, platinum, palladium, silver-silver chloride, organic electrically conductive substances, and nano-carbon materials.

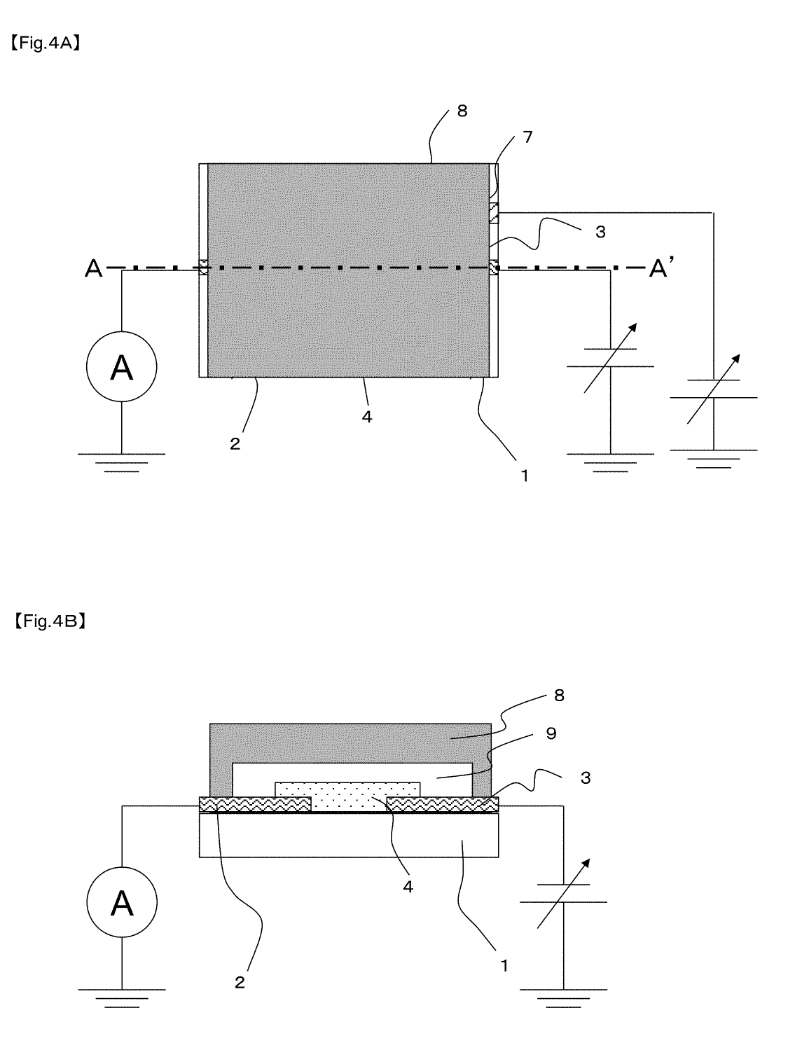

It is preferable that the sensor of the present invention further includes, on the substrate, a covering member that covers at least a portion of the substrate. For example, as a variation of the structure shown in FIG. 3, it is preferable that the sensor includes, on the substrate 1, a covering member 8 that forms an internal space with the substrate 1 as shown in FIGS. 4A and 4B. In FIG. 4A, the dotted line in the covering member 8 shows the boundary between the covering member 8 and the internal space. FIG. 4B is a cross-sectional view along the line AA′ in FIG. 4A, and an internal space 9 is shown between the substrate 1 and the covering member 8.

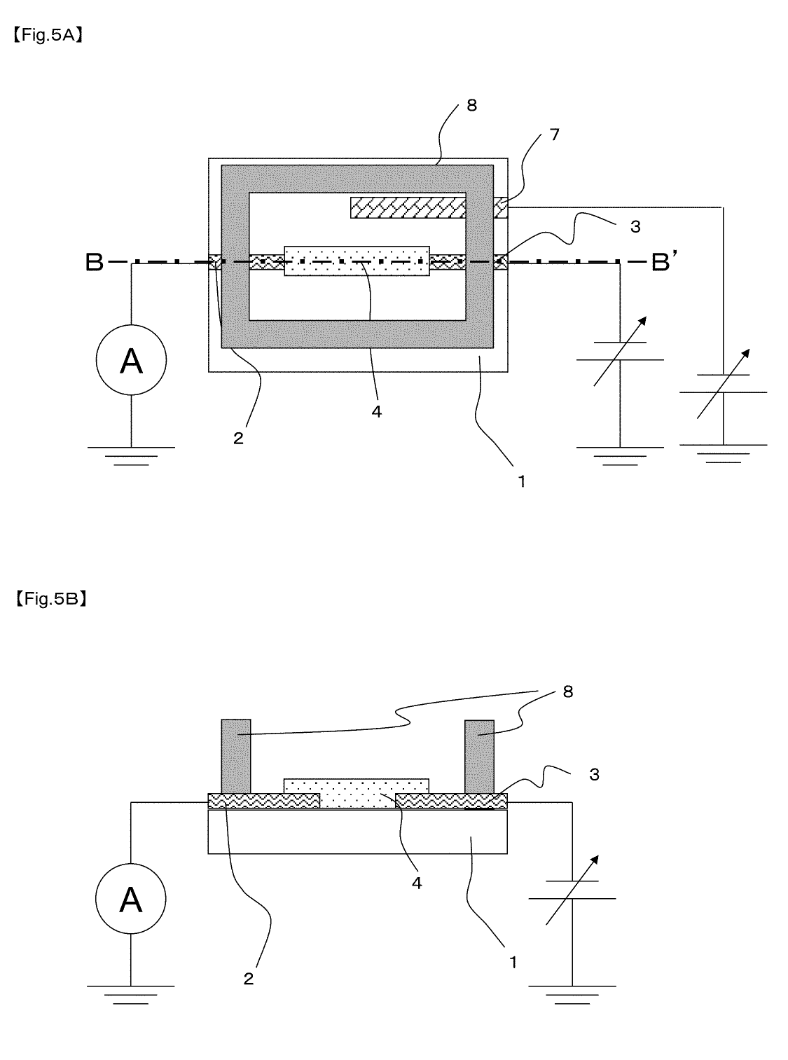

In addition, as another variation of the structure shown in FIG. 3, it is preferable that the sensor includes, on the substrate 1, a covering member 8 that forms a space 9 surrounding the semiconductor layer 4 as shown in FIGS. 5A and 5B. FIG. 5B is a cross-sectional view along the line BB′ in FIG. 5A. As a result, it becomes possible to efficiently bring the semiconductor layer 4 into contact with a liquid containing a substance to be sensed.

As another embodiment of the sensor of the present invention, it is preferable that the sensor includes the covering member described above on the substrate, and the third electrode is provided on the surface of the covering member facing the semiconductor layer. That is, it is preferable that the sensor includes a semiconductor device including a substrate, a first electrode, a second electrode, and a semiconductor layer, and further including a covering member on the substrate and a third electrode provided on the surface of the covering member facing the semiconductor layer, wherein the first electrode is spaced apart from the second electrode, the semiconductor layer is disposed between the first electrode and the second electrode, and the semiconductor layer contains the CNT composite of the present invention.

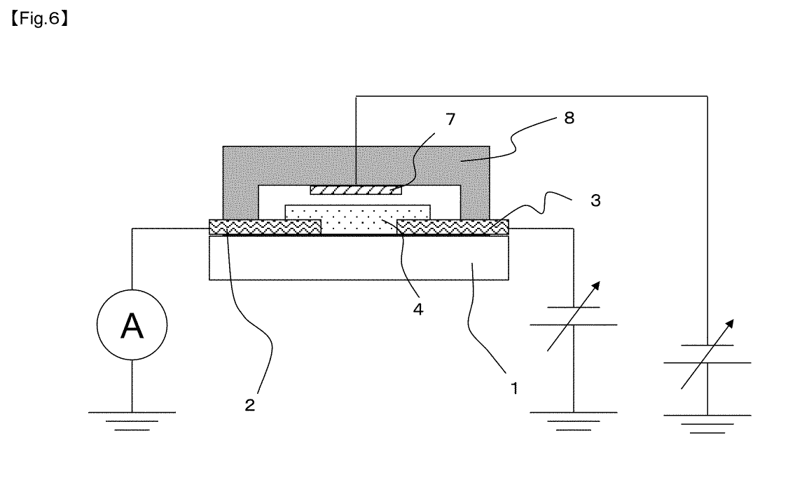

FIG. 6 is a schematic cross-sectional view showing an example of the sensor of the present invention. In the sensor of FIG. 6, a first electrode 2 and a second electrode 3 are formed on a substrate 1, and a semiconductor layer 4 is disposed between the first electrode 2 and the second electrode 3. Further, a covering member 8 is disposed on the same side as the first electrode 2, the second electrode 3, and the semiconductor layer 4 disposed on the substrate 1, and a third electrode 7 is disposed on the covering member 8. With respect to the disposition of the third electrode 7 on the covering member 8, it does not have to be disposed right above the semiconductor layer and may also be disposed diagonally thereabove. In addition, on the covering member 8, the third electrode 7 does not have to be disposed on the upper surface as seen from the semiconductor layer and also be disposed on the side surface. The third electrode 7 does not have to be disposed on the covering member 8 and may also be disposed on the substrate 1.

Examples of materials used for the covering member 8 include: inorganic materials, such as silicon wafers, glass, and alumina sintered bodies; and organic materials, such as polyimide, polyester, polycarbonate, polysulfone, polyethersulfone, polyethylene, polyphenylene sulfide, and polyparaxylene.

### EXAMPLES

Hereinafter, the present invention will be described in further detail based on examples. Incidentally, the present invention is not limited to the following examples. Incidentally, the used CNTs are as follows.

CNT 1: produced by CNI, single-wall CNTs, containing 95 wt % of semiconducting CNTs

CNT 2: produced by Meijo Nano Carbon Co., Ltd., single-wall CNTs, containing 95 wt % of metallic CNTs

In addition, of used compounds, those expressed in abbreviations are as follows.

P3HT: Poly-3-hexyl thiophene

NMP: N-methylpyrrolidone

PBS: Phosphate buffered saline

BSA: Bovine serum albumin

IgE: Immunoglobulin E

THF: Tetrahydrofuran

o-DCB: o-Dichlorobenzene

DMF: Dimethylformamide

DMSO: Dimethyl sulfoxide

SDS: Sodium dodecyl sulfate

In each example, the thickness of the blocking agent was measured using an atomic force microscope (Dimension Icon, produced by Bruker AXS).

### Example 1

(1) Production of Semiconductor Solution

1.5 mg of CNT 1 and 1.5 mg of P3HT were added to 15 ml of chloroform, and, with ice-cooling, ultrasonically stirred for 30 minutes using an ultrasonic homogenizer (VCX-500 manufactured by Tokyo Rikakikai Co., Ltd.) with an output of 250 W, thereby giving a CNT dispersion A (CNT composite concentration relative to the solvent: 0.1 g/l).

Next, a semiconductor solution for forming a semiconductor layer was produced. The CNT dispersion A was filtered using a membrane filter (pore size: 10 μm, diameter: 25 mm, Omnipore Membrane produced by Millipore) to remove CNT composites having a length of 10 μm or more. 45 ml of o-DCB was added to 5 ml of the obtained filtrate to give a semiconductor solution A (CNT composite concentration relative to the solvent: 0.01 g/l).

(2) Production of Semiconductor Device

A semiconductor device shown in FIG. 3 was produced. On a substrate 1 made of glass (thickness: 0.7 mm), gold was vacuum-deposited to a thickness of 50 nm, and a photoresist (trade name “LC100-10cP”, produced by Rohm and Haas Co.) was applied thereonto by spin coating (1,000 rpm×20 seconds), followed by heat-drying at 100° C. for 10.

The produced photoresist film was patterned by exposure to light through a mask using a parallel light mask aligner (PLA-501F manufactured by Canon Inc.), then shower-developed with ELM-D (trade name, produced by Mitsubishi Gas Chemical Company, Inc.), which is a 2.38 wt % aqueous hydroxylated tetramethylammonium solution, for 70 seconds using an automatic developing device (AD-2000 manufactured by Takizawa Co., Ltd.), and washed with water for 30 seconds. Subsequently, the substrate was subjected to an etching treatment for five minutes using AURUM-302 (trade name, produced by Kanto Chemical Co., Inc.) and then washed with water for 30 seconds. The substrate was immersed in AZ remover 100 (trade name, produced by AZ Electronic Materials) for five minutes to strip the resist, washed with water for 30 seconds, and then heat-dried at 120° C. for 20 minutes, thereby forming a first electrode 2, a second electrode 3, and a third electrode 7.

The width (channel width) of the first electrode 2 and that of the second electrode 3 were each 100 μm, and the spacing (channel length) between the first electrode 2 and the second electrode 3 was 10 μm. The third electrode 7 was disposed parallel to the second electrode 3. The spacing between the third electrode 7 and the second electrod 3 was 5 mm. Using an ink jet device (manufactured by Cluster Technology Co. , Ltd.), 400 pl of the semiconductor solution A produced by the method described in (1) above was dripped onto the electrode-formed substrate to form a semiconductor layer 4, and then subjected to a heat treatment on a hot plate in a nitrogen gas stream at 150° C. for 30 minutes, thereby giving a semiconductor device A.

Next, the voltage (Vg) on the third electrode 7 of the semiconductor device was changed, and the current (Id) between the first electrode 2 and the second electrode 3-the voltage (Vsd) between the first electrode 2 and the second electrode 3 characteristics at that time were measured. The measurement was performed in 100 μl of 0.01 M PBS (pH 7.2, produced by Wako Pure Chemical Industries, Ltd.) (air temperature: 20° C., humidity: 35%) using a semiconducting property evaluation system 4200-SCS (manufactured by Keithley Instruments Inc.). The on-off ratio at the time of changing Vg=0 to −1V was 5E+3.

Next, the semiconductor layer 4 was immersed in 1.0 mL of a DMF (manufactured by Wako Pure Chemical Industries) solution of 6.3 mg of pyrenebutanoic acid succinimide ester (produced by AnaSpec, Inc.) for 1 hour. Subsequently, the semiconductor layer 4 was sufficiently rinsed with DMF and DMSO (manufactured by Wako Pure Chemical Industries). Next, the semiconductor layer 4 was immersed in 1.0 mL of a DMSO solution of 10 μL of diethylene glycol bis(3-aminopropyl) ether (produced by Tokyo Chemical Industry Co., Ltd.) overnight. Subsequently, the semiconductor layer 4 was sufficiently rinsed with DMSO and pure water. Next, the semiconductor layer 4 was immersed in 1.0 mL of a 0.01 M PBS solution of 0.9 mg of biotin N-hydroxy sulfosuccinimide ester overnight. Subsequently, the semiconductor layer 4 was sufficiently rinsed with pure water, thereby giving a semiconductor device having biotin immobilized on the semiconductor layer 4.

The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(3) Evaluation as Sensor

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA (produced by Wako Pure Chemical Industries, Ltd.) was added to the 0.01 M PBS having immersed therein the semiconductor layer 4. Then, 20 μl of a 0.01 M PBS solution of IgE (produced by Yamasa Corporation) and 20 μl of a 0.01 M PBS solution of avidin (produced by Wako Pure Chemical Industries, Ltd.) were added thereto seven minutes and 12 minutes after the start of measurement, respectively. The results are shown in FIG. 7. The current value decreased only when avidin was added, confirming that the semiconductor device functioned as a sensor capable of specifically detecting avidin.

### Example 2

(1) Production of Semiconductor Device

A semiconductor device A was produced in the same manner as in Example 1.

Next, the semiconductor layer 4 was immersed in 1.0 mL of a DMF (manufactured by Wako Pure Chemical Industries) solution of 6.3 mg of pyrenebutanoic acid succinimide ester (produced by AnaSpec, Inc.) for 1 hour. Subsequently, the semiconductor layer 4 was sufficiently rinsed with DMF and DMSO (manufactured by Wako Pure Chemical Industries). Subsequently, the semiconductor layer 4 was immersed in 1.0 mL of a 0.01 M PBS solution of 1.5 mg of biotin hydrazide (produced by Tokyo Chemical Industry Co., Ltd.) overnight. Subsequently, the semiconductor layer 4 was sufficiently rinsed with pure water, thereby giving a semiconductor device having biotin immobilized on the semiconductor layer 4.

The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(2) Evaluation as Sensor

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA was added to the 0.01 M PBS having immersed therein the semiconductor layer 4. Then, 20 μ1 of a 0.01 M PBS solution of IgE and 20 μl of a 0.01 M PBS solution of avidin were added thereto seven minutes and 12 minutes after the start of measurement, respectively. The current value decreased by 0.05 μA only when avidin was added, confirming that the semiconductor device functioned as a sensor capable of specifically detecting avidin.

### Example 3

(1) Production of Semiconductor Device

A semiconductor device was produced in the same manner as in Example 1, except that 5.0 mL of pure water of 5.0 mg of carboxymethylcellulose (produced by Tokyo Chemical Industry Co., Ltd.) was used in place of 5.0 mL of a 0.01 M PBS solution of 5.0 mg of BSA.

(2) Evaluation as Sensor

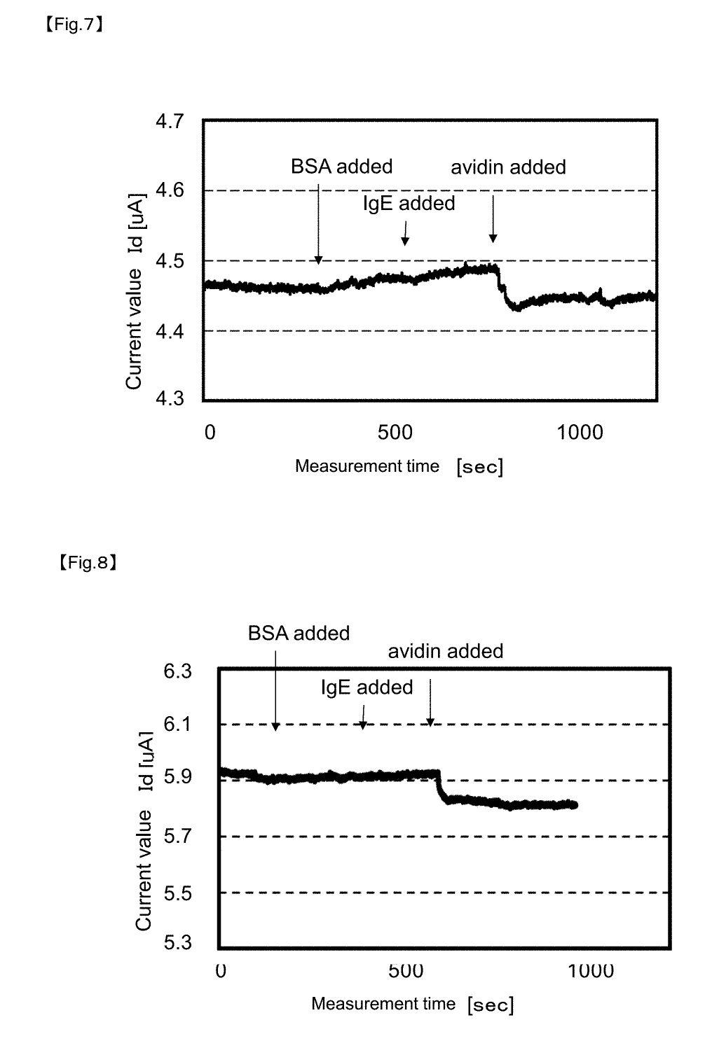

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA was added to the 0.01 M PBS having immersed therein the semiconductor layer 4. Then, 20 μl of a 0.01 M PBS solution of IgE and 20 μl of a 0.01 M PBS solution of avidin were added thereto seven minutes and 12 minutes after the start of measurement, respectively. The results are shown in FIG. 8. The current value decreased only when avidin was added, confirming that the semiconductor device functioned as a sensor capable of specifically detecting avidin.

### Example 4

(1) Production of Semiconductor Device

A semiconductor device was produced in the same manner as in Example 1, except that 5.0 mL of pure water of 5.0 mg of COATSOME NM-10 (produced by Nichiyu Corporation) was used in place of 5.0 mL of a 0.01 M PBS solution of 5.0 mg of BSA.

(2) Evaluation as Sensor

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA was added to the 0.01 M PBS having immersed therein the semiconductor layer 4. Then, 20 μl of a 0.01 M PBS solution of IgE and 20 μl of a 0.01 M PBS solution of avidin were added thereto seven minutes and 12 minutes after the start of measurement, respectively. The current value decreased by 0.1 μA only when avidin was added, confirming that the semiconductor device functioned as a sensor capable of specifically detecting avidin.

### Example 5

(1) Production of Semiconductor Solution

A CNT composite was prepared in the same manner as in Example 1, except that mixed CNTs of 1.23 mg of CNT 1 and 0.27 mg of CNT 2 were used in place of 1.5 mg of CNT 1. Then, a CNT dispersion B and a semiconductor solution B were obtained.

(2) Production of Semiconductor Device

A semiconductor device was produced in the same manner as in Example 1, except that the semiconductor solution B was used in place of the semiconductor solution A.

(3) Evaluation as Sensor

Evaluation was performed in the same manner as in Example 1. As a result, the current value decreased by 0.04 μA only when avidin was added, confirming that the semiconductor device functioned as a sensor capable of specifically detecting avidin.

### Example 6

(1) Production of Semiconductor Solution

A CNT composite was prepared in the same manner as in Example 1, except that 1.5 mg of SDS was used in place of 1.5 mg of P3HT, and 60-minute ultrasonic stirring was performed in place of 30-minute ultrasonic stirring. Then, a CNT dispersion C and a semiconductor solution C were obtained.

(2) Production of Semiconductor Device

A semiconductor device was produced in the same manner as in Example 1, except that the semiconductor solution C was used in place of the semiconductor solution A.

(3) Evaluation as Sensor

Evaluation was performed in the same manner as in Example 1. As a result, the current value decreased by 0.02 μA only when avidin was added, confirming that the semiconductor device functioned as a sensor capable of specifically detecting avidin.

### Example 7

(1) Production of Semiconductor Solution

A CNT composite was prepared in the same manner as in Example 6, except that 1.5 mg of sodium alginate was used in place of 1.5 mg of SDS. Then, a CNT dispersion D and a semiconductor solution D were obtained.

(2) Production of Semiconductor Device

A semiconductor device was produced in the same manner as in Example 1, except that the semiconductor solution D was used in place of the semiconductor solution A.

(3) Evaluation as Sensor

Evaluation was performed in the same manner as in Example 1. As a result, the current value decreased by 0.04 μA only when avidin was added, confirming that the semiconductor device functioned as a sensor capable of specifically detecting avidin.

### Example 8

(1) Production of Semiconductor Solution

A CNT composite was prepared in the same manner as in Example 6, except that 1.5 mg of sodium polystyrene sulfonate was used in place of 1.5 mg of SDS. Then, a CNT dispersion E and a semiconductor solution E were obtained.

(2) Production of Semiconductor Device

A semiconductor device was produced in the same manner as in Example 1, except that the semiconductor solution E was used in place of the semiconductor solution A.

(3) Evaluation as Sensor

Evaluation was performed in the same manner as in Example 1. As a result, the current value decreased by 0.04 μA only when avidin was added, confirming that the semiconductor device functioned as a sensor capable of specifically detecting avidin.

### Example 9

(1) Production of Semiconductor Solution

A CNT composite was prepared in the same manner as in Example 1, except that 1.5 mg of a polymer of formula (70) was used in place of 1.5 mg of P3HT. Then, a CNT dispersion F and a semiconductor solution F were obtained.

(2) Production of Semiconductor Device

A semiconductor device was produced in the same manner as in Example 1, except that the semiconductor solution F was used in place of the semiconductor solution A.

(3) Evaluation as Sensor

Evaluation was performed in the same manner as in Example 1. As a result, the current value decreased by 0.08 μA only when avidin was added, confirming that the semiconductor device functioned as a sensor capable of specifically detecting avidin.

### Example 10

(1) Production of Semiconductor Device

A semiconductor device was produced in the same manner as in Example 1, except that 5.0 mL of pure water of 5.0 mg of hexadecyltrimethylammonium bromide (produced by Nacalai Tesque) was used in place of 5.0 mL of a 0.01 M PBS solution of 5.0 mg of BSA.

(2) Evaluation as Sensor

Evaluation was performed in the same manner as in Example 1. As a result, the current value decreased by 0.08 μA only when avidin was added, confirming that the semiconductor device functioned as a sensor capable of specifically detecting avidin.

### Example 11

(1) Production of Semiconductor Device

A semiconductor device was produced in the same manner as in Example 1, except that 5.0 mL of pure water of 5.0 mg of sodium laurylphosphate (produced by Tokyo Chemical Industry Co., Ltd.) was used in place of 5.0 mL of a 0.01 M PBS solution of 5.0 mg of BSA.

(2) Evaluation as Sensor

Evaluation was performed in the same manner as in Example 1. As a result, the current value decreased by 0.08 μA only when avidin was added, confirming that the semiconductor device functioned as a sensor capable of specifically detecting avidin.

### Example 12

(1) Production of Semiconductor Device

A semiconductor device having biotin immobilized on a semiconductor layer 4 was produced in the same manner as in Example 1.

5 mL of 100 mg of 1-ethyl-3-(3-dimethylaminopropyl)carbodiimide hydrochloride (produced by DOJINDO Laboratories) was added to 5 mL of 0.01 M PBS (pH 6.0) of 100 mg of acrylic particles (produced by Corefront Corporation) and stirred at 37° C. for 2 hours. The mixture was allowed to stand, the supernatant was discarded, and then 10 mL of 0.01 M PBS (pH 6.0) was added and stirred. The mixture was allowed to stand again, and the supernatant was discarded. Subsequently, 5 mL of 0.01 M PBS (pH 6.0) was added, and 0.01 M PBS (pH 6.0) of 100 mg of BSA was further added. The mixture was stirred at 37° C. for 2 hours and then allowed to stand, and the supernatant was discarded. The operation of adding 10 mL of 0.01 M PBS, allowing the mixture to stand, and discarding the supernatant was repeated three times. 10 mL of 0.01 M PBS was added again and stirred. The above semiconductor device was immersed in 5 mL of the mixture overnight. Subsequently, the semiconductor layer 4 was sufficiently rinsed with pure water, thereby giving a semiconductor device surface-protected with a BSA/particle composite.

(2) Evaluation as Sensor

Evaluation was performed in the same manner as in Example 1. As a result, the current value decreased by 0.04 μA only when avidin was added, confirming that the semiconductor device functioned as a sensor capable of specifically detecting avidin.

### Example 13

(1) Production of Semiconductor Solution

A CNT composite was prepared in the same manner as in Example 1, except that 1.5 mg of a polymer of formula (4) was used in place of 1.5 mg of P3HT. Then, a CNT dispersion G and a semiconductor solution G were obtained.

(2) Production of Semiconductor Device

A semiconductor device G was produced in the same manner as in Example 1, except that the semiconductor solution G was used in place of the semiconductor solution A. Next, the semiconductor layer 4 was immersed in 1.0 mL of a 0.01 M PBS solution of 1.0 mg of biotin N-hydroxy sulfosuccinimide ester overnight. Subsequently, the semiconductor layer 4 was sufficiently rinsed with pure water, thereby giving a semiconductor device having biotin immobilized on the semiconductor layer 4. The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(3) Evaluation as Sensor

Evaluation was performed in the same manner as in Example 1. As a result, the current value decreased by 0.07 μA only when avidin was added, confirming that the semiconductor device functioned as a sensor capable of specifically detecting avidin.

### Example 14

(1) Production of Semiconductor Solution

A CNT composite was prepared in the same manner as in Example 1, except that 1.5 mg of a polymer of formula (46) was used in place of 1.5 mg of P3HT. Then, a CNT dispersion H and a semiconductor solution H were obtained.

(2) Production of Semiconductor Device

A semiconductor device H was produced in the same manner as in Example 1, except that the semiconductor solution H was used in place of the semiconductor solution A. Next, the semiconductor layer 4 was immersed in 1.0 mL of a 0.01 M PBS solution of 1.5 mg of biotin hydrazide overnight. Subsequently, the semiconductor layer 4 was sufficiently rinsed with pure water, thereby giving a semiconductor device having biotin immobilized on the semiconductor layer 4. The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(3) Evaluation as Sensor

Evaluation was performed in the same manner as in Example 1. As a result, the current value decreased by 0.08 μA only when avidin was added, confirming that the semiconductor device functioned as a sensor capable of specifically detecting avidin.

### Example 15

(1) Production of Semiconductor Device

A semiconductor device A was produced in the same manner as in Example 1.

The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(2) Evaluation as Sensor

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA was added to the 0.01 M PBS having immersed therein the semiconductor layer 4. Then, 20 μl of a 0.01 M PBS solution of IgE, 20 μ1 of a 0.01 M PBS solution of avidin, and 20 uL of a 0.01 M aqueous potassium phosphate (manufactured by Wako Pure Chemical Industries) solution (pH 12) were added thereto seven minutes, 12 minutes, and 17 minutes after the start of measurement, respectively. The current value decreased by 0.1 μA only when avidin was added, confirming that the semiconductor device functioned as a sensor capable of specifically detecting pH changes.

### Example 16

(1) Production of Semiconductor Device

A semiconductor device A was produced in the same manner as in Example 1.

Next, the semiconductor layer 4 was immersed in 1.0 mL of a methanol (manufactured by Wako Pure Chemical Industries) solution of 6.0 mg of pyrenebutanoic acid succinimide ester (produced by AnaSpec, Inc.) for 5 hours. Subsequently, the semiconductor layer 4 was sufficiently rinsed with a solution prepared by mixing equal volumes of methanol and water. Next, the semiconductor layer 4 was immersed in 1.0 mL of a methanol solution of 10 μL of diethylene glycol bis (3-aminopropyl) ether overnight. Subsequently, the semiconductor layer 4 was sufficiently rinsed with pure water. Next, the semiconductor layer 4 was immersed in 1.0 mL of a 0.01 M PBS solution of 0.9 mg of biotin N-hydroxy sulfosuccinimide ester overnight. Subsequently, the semiconductor layer 4 was sufficiently rinsed with pure water, thereby giving a semiconductor device having biotin immobilized on the semiconductor layer 4. The semiconductor device was immersed in 5.0 mL of a 0.01 M PBS solution of 5.0 mg of BSA overnight. Subsequently, the semiconductor layer 4 was sufficiently rinsed with pure water, thereby giving a semiconductor device surface-protected with BSA.

(2) Evaluation as Sensor

Evaluation was performed in the same manner as in Example 1. As a result, the current value decreased by 0.04 μA only when avidin was added, confirming that the semiconductor device functioned as a sensor capable of specifically detecting avidin.

### Example 17

(1) Production of Semiconductor Device

A semiconductor device was produced in the same manner as in Example 2, except that the semiconductor layer 4 was immersed in 1.0 mL of 0.01 M PBS of 100 ug/mL anti-IgE in place of 1.0 mL of a 0.01 M PBS solution of 1.5 mg of biotin hydrazide.

(2) Evaluation as Sensor

Evaluation was performed in the same manner as in Example 1. As a result, the current value decreased by 0.08 μA only when IgE was added, confirming that the semiconductor device functioned as a sensor capable of specifically detecting IgE.

### Example 18

(1) Production of Semiconductor Device

A semiconductor device was produced in the same manner as in Example 2, except that the semiconductor layer 4 was immersed in 1.0 mL of 0.01 M PBS of 100 ug/mL anti-PSA in place of 1.0 mL of a 0.01 M PBS solution of 1.5 mg of biotin hydrazide.

(2) Evaluation as Sensor

The semiconductor layer 4 of the produced semiconductor device was immersed in 100 μl of 0.01 M PBS, and the value of the current flowing between the first electrode 2 and the second electrode 3 was measured. The measurement was performed under the conditions of: the voltage (Vsd) between the first electrode and the second electrode=−0.2 V; and the voltage (Vg) between the first electrode and the third electrode=−0.6 V. Two minutes after the start of measurement, 20 μl of a 0.01 M PBS solution of BSA was added to the 0.01 M PBS having immersed therein the semiconductor layer 4. Then, 20 μ1 of a 0.01 M PBS solution of IgE and 20 μl of a 0.01 M PBS solution of PSA were added thereto seven minutes and 12 minutes after the start of measurement, respectively. The current value decreased by 0.09 μA only when PSA was added, confirming that the semiconductor device functioned as a sensor capable of specifically detecting PSA.

### Comparative Example 1

(1) Production of Semiconductor Device

A semiconductor layer 4 was formed to produce a semiconductor device in the same manner as in Example 1, except that the surface protection with BSA was not performed.

(2) Evaluation as Sensor

In order to evaluate the semiconductor device produced above as a sensor, measurement was performed in the same manner as in Example 1. Two minutes after the start of measurement, 20 μl of a 0.01 M PBS solution of BSA (produced by Wako Pure Chemical Industries, Ltd.) was added to the 0.01 M PBS having immersed therein the semiconductor layer 4. Then, 20 μl of a 0.01 M PBS solution of IgE (produced by Yamasa Corporation) and 20 μl of a 0.01 M PBS solution of avidin (produced by Wako Pure Chemical Industries, Ltd.) were added thereto seven minutes and 12 minutes after the start of measurement, respectively. BSA, IgE, and avidin were all detected, and the semiconductor device did not function as a sensor capable of specifically detecting avidin.

TABLE 1

| | | | | | Amount | | | | | | | |
|---|---|---|---|---|---|---|---|---|---|---|---|---|
| | | | | | of Or- | | | Purity | | | | |
| | | | Thick- | | ganic Com- | Bio- | | of Semi- | Semi- | | | |
| | | | ness of | | pound (C) | Re- | CNT | con- | con- | Device | | |
| | Aggre- | | Blocking | Func- | Attached | lated | Dis- | ducting | ductor | Con- | | |
| | gation | Blocking | Agent | tional | to CNTs | Mate- | per- | CNTs | Solu- | figu- | Sensing | Selec- |
| | Inhibitor | Agent | [nm] | Group | [wt %] | rial | sion | [%] | tion | ration | Object | tivity |
| Example 1 | P3HT | BSA | 3 | Amino | >95 | Biotin | A | 95 | A | FIG. 3 | BSA, | Present |
| | | | | group | | | | | | | IgE, | (avidin) |
| | | | | | | | | | | | avidin | |
| Example 2 | P3HT | BSA | 3 | Succin- | >95 | Biotin | A | 95 | A | FIG. 3 | BSA, | Present |
| | | | | imide | | | | | | | IgE, | (avidin) |
| | | | | ester | | | | | | | avidin | |
| Example 3 | P3HT | Carboxy- | 2 | Amino | >95 | Biotin | A | 95 | A | FIG. 3 | BSA, | Present |
| | | methyl- | | group | | | | | | | IgE, | (avidin) |
| | | cellulose | | | | | | | | | avidin | |
| Example 4 | P3HT | COATSOME | 2 | Amino | >95 | Biotin | A | 95 | A | FIG. 3 | BSA, | Present |
| | | NM-10 | | group | | | | | | | IgE, | (avidin) |
| | | (Sphingo- | | | | | | | | | avidin | |
| | | myelin) | | | | | | | | | | |
| Example 5 | P3HT | BSA | 3 | Amino | >95 | Biotin | B | 82 | B | FIG. 3 | BSA, | Present |
| | | | | group | | | | | | | IgE, | (avidin) |
| | | | | | | | | | | | avidin | |
| Example 6 | SDS | BSA | 3 | Amino | >95 | Biotin | C | 95 | C | FIG. 3 | BSA, | Present |
| | | | | group | | | | | | | IgE, | (avidin) |
| | | | | | | | | | | | avidin | |
| Example 7 | Sodium | BSA | 3 | Amino | >95 | Biotin | D | 95 | D | FIG. 3 | BSA, | Present |
| | alginate | | | group | | | | | | | IgE, | (avidin) |
| | | | | | | | | | | | avidin | |
| Example 8 | Sodium | BSA | 3 | Amino | >95 | Biotin | E | 95 | E | FIG. 3 | BSA, | Present |
| | polystyrene | | | group | | | | | | | IgE, | (avidin) |
| | sulfonate | | | | | | | | | | avidin | |
| Example 9 | Polymer of | BSA | 3 | Amino | >95 | Biotin | F | 95 | F | FIG. 3 | BSA, | Present |
| | formula | | | group | | | | | | | IgE, | (avidin) |
| | (70) | | | | | | | | | | avidin | |
| Example 10 | P3HT | Hexadecyl- | 2 | Amino | >95 | Biotin | A | 95 | A | FIG. 3 | BSA, | Present |
| | | trimethyl- | | group | | | | | | | IgE, | (avidin) |
| | | ammonium | | | | | | | | | avidin | |
| | | bromide | | | | | | | | | | |
| Example 11 | P3HT | Sodium | 2 | Amino | >95 | Biotin | A | 95 | A | FIG. 3 | BSA, | Present |
| | | lauryl- | | group | | | | | | | IgE, | (avidin) |
| | | phosphate | | | | | | | | | avidin | |
| Example 12 | P3HT | BSA | 46 | Amino | >95 | Biotin | A | 95 | A | FIG. 3 | BSA, | Present |
| | | | | group | | | | | | | IgE, | (avidin) |
| | | | | | | | | | | | avidin | |
| Example 13 | Polymer of | BSA | 3 | Amino | — | Biotin | G | 95 | G | FIG. 3 | BSA, | Present |
| | formula | | | group | | | | | | | IgE, | (avidin) |
| | (4) | | | | | | | | | | avidin | |
| Example 14 | Polymer of | BSA | 3 | Succin- | — | Biotin | H | 95 | H | FIG. 3 | BSA, | Present |
| | formula | | | imide | | | | | | | IgE, | (avidin) |
| | (46) | | | ester | | | | | | | avidin | |
| Example 15 | P3HT | BSA | 3 | None | — | None | A | 95 | A | FIG. 3 | BSA, | Present |
| | | | | | | | | | | | IgE, | (pH) |
| | | | | | | | | | | | pH | |
| Example 16 | P3HT | BSA | 3 | Amino | 73 | Biotin | A | 95 | A | FIG. 3 | BSA, | Present |
| | | | | group | | | | | | | IgE, | (avidin) |
| | | | | | | | | | | | avidin | |
| Example 17 | P3HT | BSA | 3 | Amino | >95 | anti- | A | 95 | A | FIG. 3 | BSA, | Present |
| | | | | group | | IgE | | | | | avidin | (IgE) |
| | | | | | | | | | | | IgE, | |
| Example 18 | P3HT | BSA | 3 | Amino | >95 | anti- | A | 95 | A | FIG. 3 | BSA, | Present |
| | | | | group | | PSA | | | | | IgE, | (PSA) |
| | | | | | | | | | | | PSA | |
| Comparative | P3HT | None | — | Amino | >95 | Biotin | A | 95 | A | FIG. 3 | BSA, | Absent |
| Example 1 | | | | group | | | | | | | IgE, | |
| | | | | | | | | | | | avidin | |

The CNT composite, semiconductor device, and sensor using the same of the present invention can be used for a wide variety of sensing applications, including chemical analysis, physical analysis, bioanalysis, and the like. The present invention is particularly suitable for use as sensor for medical use or a biosensor.

### DESCRIPTION OF REFERENCE SIGNS

1: Substrate

2: First electrode

3: Second electrode

4: Semiconductor layer

5: Gate electrode

6: Insulating layer

7: Third electrode

8: Covering member

9: Internal space

1. A carbon nanotube composite comprising an aggregation inhibitor (A) attached to at least a portion of a surface of carbon nanotubes,
the carbon nanotube composite including a blocking agent (B) attached to at least a portion of the surface of carbon nanotubes.

2. The carbon nanotube composite according to claim 1, wherein carbon nanotubes in the carbon nanotube composite include 80 wt % or more of semiconducting carbon nanotubes.

3. The carbon nanotube composite according to claim 1, wherein the aggregation inhibitor (A) is a polymer.

4. The carbon nanotube composite according to claim 3, wherein the polymer is a conjugated polymer.

5. The carbon nanotube composite according to claim 1, wherein the blocking agent (B) is selected from: a compound having at least one of a tetraalkylammonium structure and a phosphoester structure as a partial structure (B1); a polysaccharide (B2); an albumin (B3); and a phospholipid (B4).

6. A carbon nanotube composite comprising a blocking agent (B) attached to at least a portion of the surface of carbon nanotubes,
the blocking agent (B) being selected from: a compound having at least one of a tetraalkylammonium structure and a phosphoester structure as a partial structure (B1); a polysaccharide (B2); and a phospholipid (B4).

7. The carbon nanotube composite according to claim 1, wherein the blocking agent (B) has a thickness of 1 nm or more and 50 nm or less.

8. The carbon nanotube composite according to claim 1, wherein at least a portion of the aggregation inhibitor (A) or the blocking agent (B) has at least one functional group selected from a group consisting of a hydroxyl group, a carboxy group, an amino group, a mercapto group, a sulfo group, a phosphonic acid group, an organic salt or inorganic salt thereof, a formyl group, a maleimide group, and a succinimide group.

9. The carbon nanotube composite according to claim 1, comprising an organic compound (C) attached to at least a portion of the surface of carbon nanotubes, and wherein a portion of the organic compound has at least one functional group selected from the group consisting of a hydroxyl group, a carboxy group, an amino group, a mercapto group, a sulfo group, a phosphonic acid group, an organic salt or inorganic salt thereof, a formyl group, a maleimide group, and a succinimide group.

10. The carbon nanotube composite according to claim 1, wherein a bio-related material that selectively interacts with a substance to be sensed is immobilized on at least a portion of a surface.

11. A semiconductor device comprising a substrate, a first electrode, a second electrode, and a semiconductor layer,
the first electrode being spaced apart from the second electrode,
the semiconductor layer being disposed between the first electrode and the second electrode, and
the semiconductor layer containing the carbon nanotube composite according to claim 1.

12. The semiconductor device according to claim 11, wherein 70 wt % or more of the organic compound (C) is attached to the surface of carbon nanotubes.

13. A method for producing a semiconductor device comprising at least a substrate, a first electrode, a second electrode, and a semiconductor layer, the first electrode being spaced apart from the second electrode, and the semiconductor layer being disposed between the first electrode and the second electrode,
the method for producing a semiconductor device including a step of applying a solution containing the carbon nanotube composite according to claim 1, to form the semiconductor layer.

14. A method for producing a semiconductor device comprising at least a substrate, a first electrode, a second electrode, and a semiconductor layer, the first electrode being spaced apart from the second electrode, and the semiconductor layer being disposed between the first electrode and the second electrode,
the method for producing a semiconductor device including:
a step of applying a carbon nanotube composite including an aggregation inhibitor attached to at least a portion of a surface of carbon nanotubes, and then attaching a blocking agent to the carbon nanotube composite; and
a step of immobilizing a bio-related material that selectively interacts with a substance to be sensed on the carbon nanotube composite.

15. A sensor comprising the semiconductor device according to claim 11.

16. The sensor according to claim 15, further comprising a third electrode.

17. The sensor according to claim 15, further comprising, on the substrate, a covering member that covers at least a portion of the substrate.

18. The sensor according to claim 17, wherein the third electrode is provided on a surface of the covering member facing the semiconductor layer.
